SEQUENCE LISTING (25 sequences)

SEQ ID NO: 1 - protein, 97 aa, Unknown
  M2 protein (H1N1 strain) with S31N and G34E mutations
MSLLTEVETPIRNEWGCRCNDSSDPLVIAANIIEILHLILWILDRLFFKCIYRRFKYGLKRGPSTEGVPESMREEYRKEQQNAVDVDDGHFVNIELE

SEQ ID NO: 2 - protein, 97 aa, Unknown
  M2 protein (H1N1 strain) with S31 and L46P mutations
MSLLTEVETPIRNEWGCRCNDSSDPLVIAANIIGILHLILWILDRPFFKCIYRRFKYGLKRGPSTEGVPESMREEYRKEQQNAVDVDDGHFVNIELE

SEQ ID NO: 3 - protein, 97 aa, Unknown
  M2 protein (H1N1 strain)
MSLLTEVETPIRNEWGCRCNDSSDPLVIAASIIGILHLILWILDRLFFKCIYRRFKYGLKRGPSTEGVPESMREEYRKEQQNAVDVDDGHFVNIELE

SEQ ID NO: 4 - DNA, 2280 bp, Unknown
  PB2 of influenza A/Hong Kong/8/68 (H3N2) virus
atggaaagaataaaagaactacggaatctgatgtcgcagtctcgcactcgcgagatactaacaaaaaccacagttgaccatatggccataattaagaagtatacatcagggagacaggaaaagaacccgtcacttaggatgaaatggatgatggcaatgaaatatccaattacagctgacaagaggataacagaaatggttcctgagagaaatgagcaaggacaaactctatggagcaaaatgagtgatgccggatcagatcgagtgatggtatcacccttggcagtgacatggtggaatagaaatggaccaatgacaagtacggttcattatccaaaagtctacaagacttattttgagaaagtcgaaaggttaaaacatggaacctttggccctgtccattttagaaaccaagtcaaaatacgccgaagagttgacataaaccctggtcatgcagacctcagtgccaaggaggcacaagatgtaatcatggaagttgttttccccaatgaagtgggggccagaatactaacgtcggaatcacaattaacaataaccaaagagaaaaaagaagaactccaagattgcaaaatttctcctttgatggttgcatacatgttagagagagaacttgtccgaaaaacgagatttctcccagttgctggtggaacaagcagtgtatacatcgaagtgttacacttgactcaaggaacgtgttgggaacagatgtacactccaggtggagaagtgaggaatgatgatgttgatcaaagtctaattattgcagccaggaacatagtgagaagagcagcagtatcagcagatccactagcatctttattggagatgtgccacagcacacagattggcgggacaaggatggtggacattcttaggcagaacccaacggaagaacaagctgtggatatatgcaaagctgcaatgggactgagaatcagctcgtccttcagttttggcggattcacatttaagagaacaagcgggtcatcaatcaagagagaggaagaattgcttacgggcaatctccaaacattaaaaataagggtgcatgaggggtacgaggaattcacaatggtggggaaaagggcaacagctatactcagaaaagcaaccaggagattggttcagctgatagtgagtggaagagacgaacagtcagtagccgaagcaataattgtagccatggtgttttcacaagaagattgcatgataaaagcagttagaggtgatctgaatttcgttaacagggcaaatcagcgattgaatcccatgcatcaacttttaaggcattttcagaaagatgcgaaagtgctttttcaaaattggggaattgaacatatcgacaatgtaatggggatgattggagtattaccagacatgactccaagcacagagatgtcaatgagagggataagagtcagcaaaatgggcgtggatgaatactccagcacagagagggttgtggtgagcattgaccggtttttgagagttcgagaccaacgaggaaatgtattactatctcctgaggaggtcagtgaaacacaggggacagagaaactgacaataacttactcatcgtcaatgatgtgggagattaatggccctgagtcagtgttggtcaatacctatcagtggatcatcagaaactgggaaactgtcaaaattcaatggtctcagaatcctacaatgttatacaacaaaatggaatttgagccatttcagtctttagttcctaaggccattagaggccaatacagtggatttgttaggactctattccaacaaatgagggatgtacttgggacatttgataccacccagataataaagcttctcccctttgcagccgccccaccaaagcaaagtaggatgcagttctcttcattgactgtgaatgtgaggggatcagggatgagaatacttgtaaggggcaattctcctgtattcaactacaacaagacaacgaaaagactaacaattctcggaaaagatgctggcactttaattgaagacccagatgaaggtacatccggagtggagtcagctgttctgagagggttcctcattctgggtaaggaagatagaagatatggaccagcattaagcatcaatgaactgagtaaccttgcaaaaggagaaaaggctaatgtactaattgggcaaggagacgtggtgttggtaatgaaacgaaaacgggactctagcatacttactgacagccagacagcgaccaaaagaattcggatggccatcaattaa

SEQ ID NO: 5 - DNA, 2274 bp, Unknown
  PB1 of influenza A/Hong Kong/8/68 (H3N2) virus
atggatgtcaatccgactttacttttcttgaaagttccagcgcaaaatgccataagcaccacattcccttatactggagatcctccatacagccatggaacaggaacaggatacaccatggacacagtcaacagaacacatcaatattcagaaaaagggaagtggacaacaaacacggaaactggagcgccccaacttaacccaattgatggaccactacctgaggataatgagccaagtggatatgcacaaacagactgtgtcctggaagcaatggctttccttgaagaatcccacccagggatctttgaaaactcgtgtcttgaaacgatggaagttgttcaacaaacaagggtggacagactgacccaaggtcgtcagacctatgattggacattaaacagaaatcaaccggccgcaactgcattagccaacactatagaagtcttcagatcgaatggtctaacagctaatgagtcgggaaggctaatagatttcctcaaagatgtgatggaatcaatggataaagaggaaatggagataacaacacacttccaaagaaaaagaagagtaagagacaacatgaccaagaaaatggtcacacaaagaacaataggaaagaagaagcagagagtgaacaagagaagctatctaataagagcattaacattgaacacaatgaccaaagatgcagaaagaggtaaattaaagagaagagctattgcaacacccgggatgcaaatcagagggttcgtgtactttgttgaaactctagctaggagcatttgtgagaagcttgaacagtctggacttccagttggaggtaatgaaaagaaggccaaactggcaaatgttgtgagaaagatgatgactaattcacaagacacagagctttctttcacaattactggagacaatactaaatggaatgaaaatcaaaatcctcgaatgttcctggcgatgattacatatatcacaaaaaatcaacctgaatggttcagaaacgttctgagcatcgcacccataatgttctcaaacaaaatggcgagactagggaaaggatacatgttcgaaagtaagagcatgaagctccgaacacaaataccagcagaaatgctagcaagcattgacctaaagtatttcaatgaatcaacaagaaagaaaattgagaaaataaggcctcttctaatagatggcacagcttcattgagtcctggaatgatgatgggcatgttcaacatgctaagtacggttttaggagtctcaatcctgaatcttgggcaaaagagatacaccaaaacaacatactggtgggatggactccaatcctctgatgattttgctctcatagtgaatgcaccaaatcatgagggaatacaagcaggagtggatagattctacagaacctgcaagttagtcggaatcaatatgagcaagaagaagtcctatataaataggacaggaacatttgaattcacaagctttttctatcgctatggatttgtagccaattttagcatggagctgcccagttttggagtgtctgggattaatgagtcagctgatatgagcattggagtaacagtgataaagaacaacatgataaacaatgaccttggaccagcaacagcccagatggctcttcaactgttcatcaaggactacagatatacataccggtgccacagaggagacacacaaattcagacgaggagatcattcgagctaaagaagctgtgggagcaaacccgctcaaaggcaggactattggtttcagatggaggaccaaacttatacaatatccggaatcttcacatcccggaagtctgcttaaagtgggagctaatggatgaggactatcagggaagactttgtaatcccctgaatccatttgtcagccataaggagattgagtctgtaaacaatgctgtggtaatgccagctcatggtccagccaagagcatggaatatgacgctgttgcaactacacactcctggattcctaagaggaaccgctctatcctcaacacaagccaaaggggaattcttgaggatgaacagatgtatcagaagtgctgcaacctgttcgagaaatttttccccagtagttcatacaggagaccggttggaatttccagcatggtggaggccatggtgtctagggcccggattgatgccagaatagacttcgagtctggacggattaagaaagaagagttcgccgagatcatgaagatctgttccaccattgaagagctcagacggcaaaaatag

SEQ ID NO: 6 - DNA, 2151 bp, Unknown
  PA of influenza A/Hong Kong/8/68 (H3N2) virus
atggaagattttgtacgacaatgctttaatccgatgattgtcgaacttgcggaaaaggcaatgaaagagtatggagaggatcttaaaatcgaaacaaacaaatttgcagcaatatgcactcacttggaagtatgcttcatgtattcagattttcatttcatcaatgagcaaggcgagtcaatagtggtagaacttgatgatccaaatgcacttttgaagcacagatttgaaataatagagggaagagaccgcacaatggcctggacagtagtaaacagtatttgcaacaccacaggagctgagaaaccgaagtttctgccagatttgtatgattacaaggagaatagattcatcgagattggagtgacaaggagagaagtccacatatactaccttgaaaaggccaataaaattaaatctgagaatacacacatccacattttctcattcactggggaagaaatggccacaaaggccgactacactctcgatgaggaaagcagggctaggatcaaaaccagactattcaccataagacaagagatggccaacagaggcctctgggattcctttcgtcagtccgaaagaggcgaagaaacaattgaagaaagatttgaaatcacagggacaatgcgcaggcttgccgaccaaagtctcccgccgaacttctcctgccttgagaattttagagcctatgtggatggattcgaaccgaacggctacattgagggcaagctttctcaaatgtccaaagaagtgaatgcaaaaattgaaccttttctgaaaacaacaccaagaccaattagacttccggatgggcctccttgttttcagcggtccaaattccttctgatggatgctttaaagttaagcattgaggatccaagtcacgagggggagggaataccactatatgatgcgatcaaatgcatgagaacattttttggatggaaagaaccctatattgttaaaccacacgaaaaggggataaatccaaattatctgctgtcatggaagcaagtactggcagaactgcaggacattgaaaatgaggagaaaattccaagaactaaaaacatgaagaaaacgagtcagctaaagtgggcacttggtgagaacatggcaccagagaaggtagactttgacaactgtagagacgtaagcgatttgaagcaatatgatagtgacgaacctgaattaaggtcactttcaagctggatccagaatgagttcaacaaggcatgcgagctgaccgattcaacttggatagagctcgatgagattggagaagacgtggctccaattgaatacattgcaagcatgagaaggaattacttcacagcagaggtgtcccattgcagagccacagaatatataatgaagggggtatacattaatactgccttgcttaatgcatcctgtgcagcaatggacgatttccaactaattcccatgataagcaagtgtagaactaaagagggaaggcgaaagaccaatttatatggcttcatcataaaaggaagatctcacttaaggaatgacaccgacgtggtaaactttgtgagcatggagttttctctcactgacccgagacttgagccacacaaatgggagaaatactgtgtccttgagataggagatatgctactaagaagtgctataggccagatgtcaaggcctatgttcttgtatgtgagaacaaatggaacatcaaagattaaaatgaaatggggaatggagatgaggcgttgcctccttcagtcactccaacaaatcgagagtatgattgaagcagagtcatctgtcaaagagaaagacatgaccaaagagttttttgagaataaatcagaaacatggcccattggggagtcccccaagggagtggaagatggttccattgggaaggtctgcaggactttattggccaagtcggtattcaatagcctgtatgcatccccgcaattggaagggttttcagctgagtcaagaaaactgcttcttgtcgttcaggctcttaaggacaatcttgaacctggaacctttgatcttgaggggctatatgaagcaattgaggagtgcctgattaatgatccctgggttttgcttaatgcgtcgtggttcaactccttcctaacacatgcattaagatag

SEQ ID NO: 7 - DNA, 1701 bp, Unknown
  HA of influenza A/Hong Kong/8/68 (H3N2) virus
atgaagaccatcattgctttgagctacattttctgtctggctctcggccaagaccttccaggaaatgacaacagcacagcaacgctgtgcctgggacatcatgcggtgccaaacggaacactagtgaaaacaatcacagatgatcagattgaagtgactaatgctactgagctagttcagagctcctcaacggggaaaatatgcaacaatcctcatcgaatccttgatggaatagactgcacactgatagatgctctattgggggaccctcattgtgatgtttttcaaaatgagacatgggaccttttcgttgaacgcagcaaagctttcagcaactgttacccttatgatgtgccagattatgcctcccttaggtcactagttgcctcgtcaggcactctggagtttatcactgagggtttcacttggactggggtcactcagaatgggggaagcaatgcttgcaaaaggggacctggtagcggttttttcagtagactgaactggttgaccaaatcaggaagcacatatccagtgctgaacgtgactatgccaaacaatgacaattttgacaaactatacatttggggggttcaccacccgagcacgaaccaagaacaaaccagcctgtatgttcaagcatcagggagagtcacagtctctaccagaagaagccagcaaactataatcccgaatatcgggtccagaccctgggtaaggggtctgtctagtagaataagcatctattggacaatagttaagccgggagacgtactggtaattaatagtaatgggaacctaatcgctcctcggggttatttcaaaatgcgcactgggaaaagctcaataatgaggtcagatgcacctattgatacctgtatttctgaatgcatcactccaaatggaagcattcccaatgacaagccctttcaaaacgtaaacaagatcacatatggagcatgccccaagtatgttaagcaaaacaccctgaagttggcaacagggatgcggaatgtaccagagaaacaaactagaggcctattcggcgcaatagcaggtttcatagaaaatggttgggagggaatgatagacggttggtacggtttcaggcatcaaaattctgagggcacaggacaagcagcagatcttaaaagcactcaagcagccatcgaccaaatcaatgggaaattgaacagggtaatcgagaagacgaacgagaaattccatcaaatcgaaaaggaattctcagaagtagaagggagaattcaggacctcgagaaatacgttgaagacactaaaatagatctctggtcttacaatgcggagcttcttgtcgctctggagaatcaacatacaattgacctgactgactcggaaatgaacaagctgtttgaaaaaacaaggaggcaactgagggaaaatgctgaagacatgggcaatggttgcttcaaaatataccacaaatgtgacaacgcttgcatagagtcaatcagaaatgggacttatgaccatgatgtatacagagacgaagcattaaacaaccggtttcagatcaaaggtgttgaactgaagtctggatacaaagactggatcctgtggatttcctttgccatatcatgctttttgctttgtgttgttttgctggggttcatcatgtgggcctgccagagaggcaacattaggtgcaacatttgcatttga

SEQ ID NO: 8 - DNA, 1497 bp, Unknown
  NP of influenza A/Hong Kong/8/68 (H3N2) virus
atggcgtcccaaggcaccaaacggtcttatgaacagatggaaactgatggggaacgccagaatgcaactgagatcagagcatccgtcgggaagatgattgatggaattggacgattctacatccaaatgtgcactgaacttaaactcagtgattatgaggggcgactgatccagaacagcttaacaatagagagaatggtgctctctgcttttgacgaaagaaggaataaatatctggaagaacatcccagcgcggggaaggatcctaagaaaactggaggacccatatacaagagagtagatggaaagtggatgagggaactcgtcctttatgacaaagaagaaataaggcgaatctggcgccaagccaataatggtgatgatgcaacagctggtctgactcacatgatgatctggcattccaatttgaatgatacaacataccagaggacaagagctcttgttcgcaccggcatggatcccaggatgtgctctctgatgcagggttcgactctccctagaaggtctggagctgcaggcgctgcagtcaaaggagttgggacaatggtgatggagttgataaggatgatcaaacgtgggatcaatgatcggaacttctggagaggtgaaaatggacgaaaaacaaggagtgcttacgagagaatgtgcaacattctcaaaggaaaatttcaaacagctgcacaaagggcaatgatggatcaagtgagagaaagtcggaacccaggaaatgctgagatcgaagatctcatctttctggcacggtctgcactcatattgagagggtcagttgctcacaaatcttgtctgcccgcctgtgtgtatggacctgccgtagccagtggctacgacttcgaaaaagagggatactctttagtgggaatagaccctttcaaactgcttcaaaacagccaagtatacagcctaatcagaccgaacgagaatccagcacacaagagtcagctggtgtggatggcatgcaattctgctgcatttgaagatctaagagtattaagcttcatcagagggaccaaagtatccccaagggggaaactttccactagaggagtacaaattgcttcaaatgaaaacatggatgctatggaatcaagtactcttgaactgagaagcaggtactgggccataagaaccagaagtggaggaaacactaatcaacagagggcctctgcaggtcaaatcagtgtgcaacctgcattttctgtgcaaagaaacctcccatttgacaaaccaaccatcatggcagcattcactgggaatacagagggaagaacatcagacatgagggcagaaattataaggatgatggaaggtgcaaaaccagaagaaatgtccttccaggggcggggagtcttcgagctctcggacgaaaaggcagcgaacccgatcgtgccctcttttgacatgagtaatgaaggatcttatttcttcggagacaatgcagaggagtacgacaattaa

SEQ ID NO: 9 - DNA, 1410 bp, Unknown
  NA of influenza A/Hong Kong/8/68 (H3N2) virus
atgaatccaaatcaaaagataataacaattggctctgtctctctcaccattgcaacagtatgcttcctcatgcagattgccatcctggtaactactgtaacattgcattttaagcaatatgagtgcgactcccccgcgagcaaccaagtaatgccgtgtgaaccaataataatagaaaggaacataacagagatagtgtatttgaataacaccaccatagagaaagagatatgccccaaagtagtggaatacagaaattggtcaaagccgcaatgtcaaattacaggatttgcacctttttctaaggacaattcaatccggctttctgctggtggggacatttgggtgacgagagaaccttatgtgtcatgcgatcatggcaagtgttatcaatttgcactcgggcaggggaccacactagacaacaaacattcaaatgacacaatacatgatagaatccctcatcgaaccctattaatgaatgagttgggtgttccatttcatttaggaaccaggcaagtgtgtatagcatggtccagctcaagttgtcacgatggaaaagcatggctgcatgtttgtatcactggggatgacaaaaatgcaactgctagcttcatttatgacgggaggcttgtggacagtattggttcatggtctcaaaatatcctcagaacccaggagtcggaatgcgtttgtatcaatgggacttgcacagtagtaatgactgatggaagtgcttcaggaagagccgatactagaatactattcattgaagaggggaaaattgtccatattagcccattgtcaggaagtgctcagcatgtagaagagtgttcctgttatcctagatatcctggcgtcagatgtatctgcagagacaactggaaaggctctaataggcccgtcgtagacataaatatggaagattatagcattgattccagttatgtgtgctcagggcttgttggcgacacacctagaaacgacgacagatctagcaatagcaattgcaggaatcctaacaatgagagagggaatcaaggagtgaaaggctgggcctttgacaatggagatgacgtgtggatgggaagaacgatcagcaaggatttacgctcaggttatgaaactttcaaagtcattggtggttggtccacacctaattccaaatcgcagatcaatagacaagtcatagttgacagcgataatcggtcaggttactctggtattttctctgttgagggcaaaagctgcatcaataggtgcttttatgtggagttgataaggggaaggaaacaggagactagagtgtggtggacctcaaacagtattgttgtgttttgtggcacttcaggtacctatggaacaggctcatggcctgatggggcgaacatcaatttcatgcctatataa

SEQ ID NO: 10 - DNA, 982 bp, Unknown
  M of influenza A/Hong Kong/8/68 (H3N2) virus
atgagccttctaaccgaggtcgaaacgtacgttctctctatcgtcccgtcaggccccctcaaagccgagatcgcacagagacttgaagatgtctttgctgggaagaacacagatcttgaggctctcatggaatggctaaagacaagaccaatcctgtcacctctgactaaggggattttgggatttgtattcacgctcaccgtgcccagtgagcgaggactgcagcgtagacgctttgtccaaaatgccctcaatgggaatggggatccaaataacatggacagagcagttaaactgtatagaaaacttaagagggagataacattccatggggccaaagaaatagcactcagttattctgctggtgcacttgccagttgcatgggcctcatatacaacaggatgggggctgtgaccactgaagtggcctttggcctggtatgtgcaacctgtgaacagattgctgactcccagcataggtctcataggcaaatggtgacaacaaccaatccactaataagacatgagaacagaatggttctggccagcactacagctaaggctatggagcaaatggctggatcgagtgagcaggcagcagaggccatggaggttgctagtcaggccaggcaaatggtgcaggcaatgagagccattgggactcatcctagctccagtgctggtctaaaagatgatcttcttgaaaatttgcaggcctatcagaaacgaatgggggtgcagatgcaacgattcaagtgaccctcttgttgttgctgcgagtatcatcgggatcttgcacttgatattgtggattcttgatcgtctttttttcaaatgcatttatcgattctttgaacacggtctgaaaagagggccttctacggaaggagtacctgagtctatgagggaagaatatcgaaaggaacagcagagtgctgtggatgctgacgatagtcattttgtcagcatagagctggagtaa

SEQ ID NO: 11 - DNA, 838 bp, Unknown
  NS of influenza A/Hong Kong/8/68 (H3N2) virus
atggattctaacactgtgtcaagttttcaggtagattgcttcctttggcatgtccgaaaacaagttgtagaccaagaactaggtgatgccccattccttgatcggcttcgccgagatcagaagtccctaaggggaagaggcagcactctcggtctaaacatcgaagcagccacccgtgttggaaagcagatagtagagaggattctgaaggaagaatccgatgaggcacttaaaatgaccatggcctccgcacctgcttcgcgatacctaactgacatgactattgaggaattgtcaagggactggttcatgctaatgcccaagcagaaagtggaaggacctctttgcatcagaatagaccaggcaatcatggataagaacatcatgttgaaagcgaatttcagtgtgatttttgaccggctagagaccctaatattactaagggctttcaccgaagagggagcaattgttggcgaaatctcaccattgccttctcttccaggacatactattgaggatgtcaaaaatgcaattggggtcctcatcggaggacttgaatggaatgataacacagttcgagtctctaaaactctacagagattcgcttggggaagcagtaatgagaatgggagacctccactcactccaaaacagaaacggaaaatggcgagaacagttaggtcaaaagttcgaagagataagatggctgattgaagaagtgagacacagattgaagacaacagagaatagttttgagcaaataacatttatgcaagccttacagctactatttgaagtggaacaggagataagaactttctcgtttcagcttatttaa

SEQ ID NO: 12 - DNA, 1698 bp, Unknown
  HA of influenza A/WSN/33 (H1N1) virus
atgaaggcttttgtactagtcctgttatatgcatttgtagctacagatgcagacacaatatgtataggctaccatgcgaacaactcaaccgacactgttgacacaatattcgagaagaatgtggcagtgacacattctgttaacctgctcgaagacagacacaacgggaaactatgtaaattaaaaggaatagccccactacaattggggaaatgtaacatcaccggatggctcttgggaaatccagaatgcgactcactgcttccagcgagatcatggtcctacattgtagaaacaccaaactctgagaatggagcatgttatccaggagatttcatcgactatgaggaactgagggagcaattgagctcagtatcatcattagaaagattcgaaatatttcccaaggaaagttcatggcccaaccacacattcaacggagtaacagtatcatgctcccataggggaaaaagcagtttttacagaaatttgctatggctgacgaagaagggggattcatacccaaagctgaccaattcctatgtgaacaataaagggaaagaagtccttgtactatggggtgttcatcacccgtctagcagtgatgagcaacagagtctctatagtaatggaaatgcttatgtctctgtagcgtcttcaaattataacaggagattcaccccggaaatagctgcaaggcccaaagtaaaagatcaacatgggaggatgaactattactggaccttgctagaacccggagacacaataatatttgaggcaactggtaatctaatagcaccatggtatgctttcgcactgagtagagggtttgagtccggcatcatcacctcaaacgcgtcaatgcatgagtgtaacacgaagtgtcaaacaccccagggatctataaacagcaatctccctttccagaatatacacccagtcacaataggagagtgcccaaaatatgtcaggagtaccaaattgaggatggttacaggactaagaaacatcccatccattcaatacagaggtctatttggagccattgctggttttattgaggggggatggactggaatgatagatggatggtatggttatcatcatcagaatgaacagggatcaggctatgcagcggatcaaaaaagcacacagaatgccattaacaggattacaaacaaggtgaactctgttatcgagaaaatgaacactcaattcacagctgtgggtaaagaattcaacaacttagaaaaaaggatggaaaatttaaataaaaaagttgatgatgggtttctggacatttggacatataatgcagaattgttagttctactggaaaatgaaagaactttggatttccatgacttaaatgtgaagaatctgtacgagaaagtaaaaagccaattaaagaataatgccaaagaaatcggaaatgggtgttttgagttctaccacaagtgtgacaatgaatgcatggaaagtgtaagaaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatctga

SEQ ID NO: 13 - DNA, 819 bp, Unknown
  M of influenza A/WSN/33 (H1N1) virus
atgagtcttctaaccgaggtcgaaacgtacgttctctctatcgtcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacgaagatccaaataacatggacaaagcagttaaactgtgtaggaagcttaagagggagataacattccatggggccaaagaaatagcactcagttattctgctggtgcacttgccagttgtatgggcctcatatacaacaggataggggctgtgaccactgaagtggcatttggcctggtatgcgcaacctgtgaacagattgctgactcccagcatcggtctcataggcaaatggtgacaacaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccattgggactcatcctagctccagtgctggtctaaaagatgatcttcttgaaaatttgcaggcctatcagaaacgaatgggggtgcagatgcaacgattcaagtgatcctctcgtcattgcagcaaatatcattggaatcttgcacttgatattgtggattcttga

SEQ ID NO: 14 - DNA, 1368 bp, Unknown
  NA of influenza A/WSN/33 (H1N1) virus
atgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcagcattacctataaagttgttgctgggcaggactcaacttcagtgatattaaccggcaattcatctctttgtcccatccgtgggtgggctatacacagcaaagacaatggcataagaattggttccaaaggagacgtttttgtcataagagagccttttatttcatgttctcacttggaatgcaggaccttttttctgactcaaggcgccttactgaatgacaagcattcaagggggacctttaaggacagaagcccttatagggccttaatgagctgccctgtcggtgaagctccgtccccgtacaattcaaggtttgaatcggttgcttggtcagcaagtgcatgtcatgatggaatgggctggctaacaatcggaatttctggtccagatgatggagcagtggctgtattaaaatacaacggcataataactgaaaccataaaaagttggaggaagaatatattgagaacacaagagtctgaatgtacctgtgtaaatggttcatgttttaccataatgaccgatggcccaagtgatgggctggcctcgtacaaaattttcaagatcgagaaggggaaggttactaaatcaatagagttgaatgcacctaattctcactacgaggaatgttcctgttaccctgataccggcaaagtgatgtgtgtgtgcagagacaattggcacggttcgaaccgaccatgggtgtccttcgaccaaaacctagattataaaataggatacatctgcagtggggttttcggtgacaacccgcgtcccaaagatggaacaggcagctgtggcccagtgtctgctgatggagcaaacggagtaaagggattttcatataagtatggtaatggtgtttggataggaaggactaaaagtgacagttccagacatgggtttgagatgatttgggatcctaatggatggacagagactgatagtaggttctctatgagacaagatgttgtggcaatgactgatcggtcagggtacagcggaagtttcgttcaacatcctgagctaacagggctagactgtatgaggccttgcttctgggttgaattaatcagggggctacctgaggagaacgcaatctggactagtgggagcatcatttctttttgtggtgtgaatagtgatactgtagattggtcttggccagacggtgctgagttgccgttcaccattgacaagtagtttgtt

SEQ ID NO: 15 - DNA, 1501 bp, Unknown
  NP of influenza A/WSN/33 (H1N1) virus
atggcgaccaaaggcaccaaacgatcttacgaacagatggagactgatggagaacgccagaatgccactgaaatcagagcatctgtcggaaaaatgattgatggaattggacgattctacatccaaatgtgcaccgaacttaaactcagtgattatgagggacggctgattcagaacagcttaacaatagagagaatggtgctctctgcttttgacgagaggaggaataaatatctagaagaacatcccagtgcggggaaagatcctaagaaaactggaggacctatatacaggagagtagatggaaagtggaggagagaactcatcctttatgacaaagaagaaataagacgaatctggcgccaagctaataatggtgacgatgcaacggctggtctgactcacatgatgatctggcactccaatttgaatgatgcaacttaccagaggacaagagctcttgttcgcacaggaatggatcccaggatgtgctcactgatgcagggttcaaccctccctaggaggtctggggccgcaggtgctgcagtcaaaggagttggaacaatggtgatggaattgatcagaatgatcaaacgtgggatcaatgatcggaacttctggaggggtgagaatggacggagaacaaggattgcttatgaaagaatgtgcaacattctcaaagggaaatttcaaacagctgcacaaagaacaatggtggatcaagtgagagagagccggaatccaggaaatgctgagttcgaagatctcatctttttagcacggtctgcactcatattgagagggtcagttgctcacaagtcctgcctgcctgcctgtgtgtatggatctgccgtagccagtggatacgactttgaaagagagggatactctctagtcggaatagaccctttcagactgcttcaaaacagccaagtatacagcctaatcagaccaaatgagaatccagcacacaagagtcaactggtgtggatggcatgccattctgctgcatttgaagatctaagagtatcaagcttcatcagagggacgaaagtggtcccaagagggaagctttccactagaggagttcaaattgcttccaatgaaaacatggagactatggaatcaagtacccttgaactgagaagcagatactgggccataaggaccagaagtggagggaacaccaatcaacagagggcttcctcgggccaaatcagcatacaacctacgttctcagtacagagaaatctcccttttgacagaccaaccattatggcagcattcactgggaatacagaggggagaacatctgacatgagaaccgaaatcataaggctgatggaaagtgcaagaccagaagatgtgtctttccaggggcggggagtcttcgagctctcggacgaaaaggcaacgagcccgatcgtgccctcctttgacatgagtaatgaaggatcttatttcttcggagacaatgcagaggagtacgacaattaaagaa

SEQ ID NO: 16 - DNA, 693 bp, Unknown
  NS of influenza A/WSN/33 (H1N1) virus
atggatccaaacactgtgtcaagctttcaggtagattgctttctttggcatgtccgcaaaagagttgcagaccaagaactaggtgatgccccattccttgatcggcttcgccgagatcagaagtccctaagaggaagaggcagcactctcggtctggacatcgaaacagccacccgtgctggaaagcaaatagtggagcggattctgaaggaagaatctgatgaggcactcaaaatgaccatggcctctgtacctgcatcgcgctacctaactgacatgactcttgaggaaatgtcaaggcactggttcatgctcatgcccaagcagaaagtggcaggccctctttgtatcagaatggaccaggcgatcatggataagaacatcatactgaaagcgaacttcagtgtgatttttgaccggctggagactctaatattactaagggccttcaccgaagaggggacaattgttggcgaaatttcaccactgccctctcttccaggacatactgatgaggatgtcaaaaatgcagttggggtcctcatcggaggacttgaatggaataataacacagttcgagtctctgaaactctacagagattcgcttggagaagcagtaatgagaatgggagacctccactcactccaaaacagaaacggaaaatggcgggaacaattaggtcagaagtttga

SEQ ID NO: 17 - DNA, 2151 bp, Unknown
  PA of influenza A/WSN/33 (H1N1) virus
atggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaattttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactggaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatag

SEQ ID NO: 18 - DNA, 2274 bp, Unknown
  PB1 of influenza A/WSN/33 (H1N1) virus
atggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagattgtgtattggaagcaatggccttccttgaggaatcccatcctggtatctttgagacctcgtgtcttgaaacgatggaggttgttcagcaaacacgagtggacaagctgacacaaggccgacagacctatgactggactctaaataggaaccagcctgctgcaacagcattggccaacacaatagaagtgttcagatcaaatggcctcacggccaatgaatctggaaggctcatagacttccttaaggatgtaatggagtcaatgaacaaagaagaaatggagatcacaactcattttcagagaaagagacgagtgagagacaatatgactaagaaaatggtgacacagagaacaataggtaaaaggaagcagagattgaacaaaaggagttatctaattagggcattaaccctgaacacaatgaccaaagatgctgagagagggaagctaaaacggagagcaattgcaaccccagggatgcaaataagggggtttgtatactttgttgagacactagcaaggagtatatgtgagaaacttgaacaatcaggattgccagttggaggcaatgagaagaaagcaaagttggcaaatgttgtaaggaagatgatgaccaattctcaggacactgaaatttctttcaccatcactggagataacaccaaatggaacgaaaatcagaaccctcggatgtttttggccatgatcacatatataaccagaaatcagcccgaatggttcagaaatgttctaagtattgctccaataatgttctcaaacaaaatggcgagactgggaaaggggtacatgtttgagagcaagagtatgaaacttagaactcaaatacctgcagaaatgctagcaagcatcgatttgaaatacttcaatgattcaactagaaagaagattgaaaaaatccggccgctcttaatagatgggactgcatcattgagccctggaatgatgatgggcatgttcaatatgttaagtactgtattaggcgtctccatcctgaatcttggacaaaagagacacaccaagactacttactggtgggatggtcttcaatcttctgatgattttgctctgattgtgaatgcacccaatcatgaagggattcaagccggagtcaacaggttttatcgaacctgtaagctacttggaattaatatgagcaagaaaaagtcttacataaacagaacaggtacatttgaattcacaagttttttctatcgttatgggtttgttgccaatttcagcatggagcttcccagctttggggtgtctgggatcaacgagtctgcggacatgagtattggagttactgtcatcaaaaacaatatgataaacaatgatcttggtccagcaaccgctcaaatggcccttcagctgttcatcaaagattacaggtacacgtaccggtgccatagaggtgacacacaaatacaaacccgaagatcatttgaaataaagaaactgtgggagcaaacccattccaaagctggactgctggtctccgacggaggcccaaatttatacaacattagaaatctccacattcctgaagtctgcttgaaatgggaattaatggatgaggattaccaggggcgtttatgcaacccactgaacccatttgtcaaccataaagacattgaatcagtgaacaatgcagtgataatgccagcacatggtccagccaaaaacatggagtatgatgctgttgcaacaacacactcctggatccccaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaagatctgttccaccattgaagagctcagacggcaaaaatag

SEQ ID NO: 19 - DNA, 2280 bp, Unknown
  PB2 of influenza A/WSN/33 (H1N1) virus
atggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaacctttggccctgtccattttagaaaccaagtcaaaatacgtcgaagagttgacataaatcctggtcatgcagatctcagtgccaaagaggcacaggatgtaatcatggaagttgttttccctaacgaagtgggagccaggatactaacatcggaatcgcaactaacgacaaccaaagagaagaaagaagaactccagggttgcaaaatttctcctctgatggtggcatacatgttggagagagaactggtccgcaaaacgagattcctcccagtggctggtggaacaagcagtgtgtacattgaagtgttgcatttgacccaaggaacatgctgggaacagatgtacactccaggaggggaggcgaggaatgatgatgttgatcaaagcttaattattgctgctagaaacatagtaagaagagccacagtatcagcagatccactagcatctttattggagatgtgccacagcacgcagattggtggagtaaggatggtaaacatccttaggcagaacccaacagaagagcaagccgtggatatttgcaaggctgcaatgggactgagaattagctcatccttcagttttggtggattcacatttaagagaacaagcggatcatcagtcaagagagaggaagaggtgcttacgggcaatcttcagacattgaagataagagtgcatgagggatatgaagagttcacaatggttgggagaagagcaacagctatactcagaaaagcaaccaggagattgattcagctgatagtgagtgggagagacgaacagtcgattgccgaagcaataattgtggccatggtattttcacaagaggattgtatgataaaagcagttagaggtgacctgaatttcgtcaatagggcgaatcagcgattgaatcccatgcaccaacttttgagacattttcagaaggatgcaaaggtgctctttcaaaattggggaattgaatccatcgacaatgtgatgggaatgatcgggatattgcccgacatgactccaagcaccgagatgtcaatgagaggagtgagaatcagcaaaatgggggtagatgagtattccagcgcggagaagatagtggtgagcattgaccgttttttgagagttagggaccaacgtgggaatgtactactgtctcccgaggaggtcagtgaaacacagggaacagagaaactgacaataacttactcatcgtcaatgatgtgggagattaatggtcctgaatcagtgttggtcaatacctatcagtggatcatcagaaactgggaaactgttaaaattcagtggtcccagaatcctacaatgctgtacaataaaatggaatttgagccatttcagtctttagttccaaaggccgttagaggccaatacagtgggtttgtgagaactctgttccaacaaatgagggatgtgcttgggacatttgataccgctcagataataaaacttcttcccttcgcagccgctccaccaaagcaaagtggaatgcagttctcctcattgactataaatgtgaggggatcaggaatgagaatacttgtaaggggcaattctccagtattcaactacaacaagaccactaaaagactcacagttctcggaaaggatgctggccctttaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtgttggtaatgaaacggaaacggaactctagcatacttactgacagccagacagcgaccaaaagaattcggatggccatcaattag

SEQ ID NO: 20 - DNA, 72 bp, Unknown
  M2 protein (H1N1 strain)
cctctcgtcattgcagcaagtatcattggaatcttgcacttgatattgtggattcttgatcgtctttttttc

SEQ ID NO: 21 - DNA, 10 bp, Unknown
  M2 protein (H1N1 strain) with S31N mutation
gcaaatatca

SEQ ID NO: 22 - DNA, 19 bp, Unknown
  M2 protein (H1N1 strain) with S31N and G34E mutation
gcaaatatcattgaaatct

SEQ ID NO: 23 - DNA, 60 bp, Unknown
  M2 protein (H1N1 strain) with S31N and L46P mutation
gcagcaaatatcattggaatcttgcacttgatattgtggattcttgatcgtccttttttc

SEQ ID NO: 24 - DNA, 10 bp, Unknown
  Oseltamivir-resistant influenza virus
tctcactacg

SEQ ID NO: 25 - DNA, 10 bp, Unknown
  Oseltamivir-resistant influenza virus with NA-H274Y mutation
tcttactacg

(19) United States
(12) Patent Application Publication
(10) Pub. No.: US 2022/0202929 A1
(43) Pub. Date: Jun. 30, 2022

(54) DRUG-RESISTANT INFLUENZA VIRUS STRAINS

(71) Applicant: President and Fellows of Harvard College, Cambridge, MA (US)

(72) Inventors: Donald E. Ingber, Boston, MA (US); Longlong Si, Boston, MA (US); Rachelle Prantil-Baun, Ashlad, MA (US)

(73) Assignee: President and Fellows of Harvard College, Cambridge, MA (US)

(21) Appl. No.: 17/613,011

(22) PCT Filed: May 19, 2020
(86) PCT No.: PCT/US2020/033590

§ 371 Date: Nov. 19, 2021

Related U.S. Application Data
(60) Provisional application No. 62/850,113, filed on May 20, 2019.

(51) Int. Cl. A61K 39/145; C12N 7/00; A61P 31/16

(52) U.S. Cl. CPC A61K 39/145; C12N 7/00; C12N 2760/16151; C12N 2760/16121; C12N 2760/16122; A61P 31/16

(57) ABSTRACT

This disclosure provides immunogenic compositions and methods of producing immunogenic compositions sufficient to produce an antigen-specific immune response against variant influenza virus strains. Also provided herein are methods of identifying drug-resistant influenza virus strains.

### RELATED APPLICATION

This application claims the benefit under 35 U.S.C. § 119(e) of U.S. provisional application No. 62/850,113, filed May 20, 2019, which is incorporated by reference herein in its entirety.

### GOVERNMENT LICENSE RIGHTS

This invention was made with government support under HL141797 awarded by National Institutes of Health. The government has certain rights in the invention.

### BACKGROUND

Influenza is a disease caused by influenza virus infection of the respiratory tract epithelium that has a global impact, causing a high percentage of morbidity and mortality every year. Influenza pandemics in human populations due to rapid viral evolution can spread globally within months or even weeks at unpredictable intervals. Vaccine development that is initiated upon emergence of a pandemic is not sufficient to prevent or mitigate the first pandemic wave.

### SUMMARY

The present disclosure provides, in some aspects, methods of identifying influenza virus variants likely to evolve during human transmission, under the selective pressure of anti-influenza drug therapies. Being able to predict the emergence of such variants would allow the development and stockpiling of effective vaccines and other immunogenic compositions for preventing and/or treating otherwise drug-resistant strains of influenza virus. This early development and stockpiling should enable early prevention and/or containment of influenza virus infection by newly emerging variant strains, thus preventing an influenza pandemic.

Some aspects of the present disclosure provide methods comprising (a) evolving a parent strain of influenza viral particles in cell culture comprising human airway (e.g., lung) cells in the presence of an anti-influenza drug, and (b) isolating drug-resistant progeny influenza viral particles released from the human airway cells. In some embodiments, the evolving step comprises culturing human airway cells that comprise a drug-sensitive parent strain of influenza viral particles in cell culture comprising an anti-influenza drug for a period of time sufficient to inhibit viral replication and/or viral spread of at least 70% of the influenza viral particles (to reduce the influenza viral titer by at least 70%, relative to baseline (prior to expose to the drug)), and/or culturing human airway cells that comprise progeny of the influenza viral particles in cell culture comprising the anti-influenza drug.

Other aspects of the present disclosure provide methods comprising (a) culturing human airway (e.g., lung) cells that comprise a drug-sensitive parent strain of influenza viral particles in cell culture that comprises an anti-influenza drug for a period of time sufficient to inhibit viral replication and/or viral spread of a subset of the influenza viral particles (to reduce the influenza viral titer), (b) culturing human airway cells that comprise progeny of the influenza viral particles in cell culture that comprises the anti-influenza drug, and (c) isolating drug-resistant progeny influenza viral particles released from the human airway cells.

In some embodiments, the methods further comprises sequencing viral RNA obtained from the drug-resistant progeny influenza viral particles to identify a drug-resistant strain of influenza virus comprising a mutation in its genome, relative to the parent strain of influenza virus.

Also provided herein, in some aspects, are immunogenic compositions comprising an influenza virus matrix 2 (M2) antigen variant that comprises an amino acid substitution at position 31 and an amino acid substitution at position 34, relative to a H1N1 influenza virus M2 antigen, wherein the H1N1 influenza virus M2 antigen comprises the amino acid sequence of SEQ ID NO: 3. Other aspects provide immunogenic compositions comprising an influenza virus matrix 2 (M2) antigen variant that comprises an amino acid substitution at position 31 and an amino acid substitution at position 46, relative to a H1N1 influenza virus M2 antigen, wherein the H1N1 influenza virus M2 antigen comprises the amino acid sequence of SEQ ID NO: 3.

In some embodiments, the amino acid substitution at position 31 is S31N. In some embodiments, the amino acid substitution at position 34 is G34E. In some embodiments, the amino acid substitution at position 46 is L46P. In some embodiments, the influenza virus M2 antigen variant comprises the amino acid sequence of SEQ ID NO: 1. In some embodiments, the influenza virus M2 antigen variant comprises an amino acid sequence that shares at least 95% identity with the amino acid sequence of SEQ ID NO: 1. In some embodiments, the influenza virus M2 antigen variant comprises the amino acid sequence of SEQ ID NO: 2. In some embodiments, the influenza virus M2 antigen variant comprises an amino acid sequence that shares at least 95% identity with the amino acid sequence of SEQ ID NO: 2.

Further provided herein, in some embodiments, are methods comprising administering to a subject the immunogenic composition of any one of the embodiments of the present disclosure in an effective amount to induce in the subject an antigen-specific immune response (to influenza).

### BRIEF DESCRIPTION OF THE DRAWINGS

FIG. 1A shows a schematic diagram cross-section through a small airway-on-a-chip. The small airway was infected with influenza viruses through the air channel. FIG. 1B shows that on both the 0.4 μm and the 7.0 μm chip, the differentiated human small airway epithelium exhibited well-structured cilia, as demonstrated by α-tubulin staining, and continuous tight junctions, as demonstrated by ZO1 staining. The endothelium also exhibited continuous adherens junctions between adjacent cells, as indicated by VE-Cadherin staining. FIG. 1C shows that the barrier function of human small airway on chip was measured using a Cascade blue (607 Da) assay. Barrier permeability is presented as apparent permeability (Papp; data from 4 independent biological replications). FIG. 1D shows the level of mucus produced at weeks 1, 2, 3, and 4 post-differentiation as quantified using Alcian Blue assay. FIG. 1E shows the fold changes in expression level of serine proteases in the differentiated human small airway-on-a-chip versus undifferentiated human small airway cells or MDCK.2 cells.

FIG. 2A shows GFP-labeled NS plasmid and one of seven wild-type plasmids (HA, NA, PA, NP, PB1, PB2, or M) that were co-transfected into a HEK293T/MDCK.2 co-culture. FIG. 2B shows fluorescence microscopy analysis of MDCK.2 cells infected with GFP-labeled PR8 virus (MOI=0.01), in the absence or presence of anti-HA antibody (5 μg/mL). Twenty-four hours post-infection, the cells were fixed and stained with DAPI. Anti-HA antibody results in decreased GFP signal.

FIG. 3A shows influenza virus infection causes damage of continuous tight junctions, as demonstrated by ZO1 staining. The human small airway was infected by GFP-labelled influenza A/PR/8/34 (H1N1) virus (MOI=0.1), and cultured for 48 hours at 37° C. under 5% CO₂. FIG. 3B shows influenza virus infection causes damaged of endothelium, as demonstrated by VE-Cadherin staining. FIG. 3C shows a barrier function of human small airway in the presence or absence of influenza virus as measured by Cascade blue (607 Da) assay. The increased apparent permeability (Papp) value indicated the influenza virus infection decreased the barrier function of human small airway-on-a-chip. FIG. 3D shows influenza virus infection causes the damage to cilia on the epithelium of human small airway, as demonstrated by α-tubulin.

FIGS. 4A-4C show viral replication kinetics of influenza viruses in health/COPD human small airway chips infected with influenza A/WSN/33 (H1N1) virus (MOI=0.001) or influenza A/Hong Kong/8/68/(H3N2) virus (MOI=0.01), and their effects on the cilia of the epithelium of human small airway.

FIG. 5A shows plaque formation at the first and eight passage of a multi-passaging experiment on human airway-on-a-chip treated with amantadine. The human airway was infected with influenza A/WSN/33 (H1N1) (MOI=0.1) and treated with amantadine or left untreated. At 48 hours (h) post-infection, supernatants were taken and employed for infection in the next round of investigation. Virus yields of untreated human airway were arbitrarily set at 100%. FIG. 5B shows the identification of amantadine-resistant influenza virus strains by viral genome sequencing. Three classes of mutations in the M2 protein of influenza virus were identified (e.g., S31N, S31N/G34E, and S31N/L46P). FIG. 5C shows the activity of amantadine against parent influenza virus strains and the S31N, S31N/G34E, and S31N/L46P mutant influenza virus strains.

FIG. 6A shows plaque formation at the first and twenty-fifth passage of a multi-passaging experiment on human airway-on-a-chip treated with oseltamivir. The human airway was infected with influenza A/WSN/33 (H1N1) (MOI=0.1) and treated with oseltamivir or left untreated. At 48 h post-infection, supernatants were taken and employed for infection in the next round of investigation. Virus yields of mock-treated human airway were arbitrarily set at 100%.

FIG. 6B shows the identification of oseltamivir-resistant influenza virus strains by viral genome sequencing. One class of mutant was identified (e.g., NA-H274Y). The mutation in the mutant occurred in the Neuraminidase A (NA) protein of the influenza virus. FIG. 6C shows the activity of oseltamivir against parent influenza virus strain and the H274Y mutant influenza virus strain.

FIG. 7 shows the genotypes of influenza virus reassortants isolated from human airway co-infected by influenza A/WSN/33 (H1N1) virus (MOI=0.01) and influenza A/Hong Kong/8/68 (H3N2) virus (MOI=0.01).

### DETAILED DESCRIPTION

One of the greatest challenges for prevention and treatment of influenza virus is the rapid rate at which the virus evolves as it spreads through human populations. The accumulation of mutations in the viral genome is responsible for influenza antigenic shift over time, which results in the emergence of new influenza virus strains, limiting the effectiveness of current anti-influenza drugs and vaccines. Thus, inhibiting the ability of influenza virus to rapidly change is a major challenge for the design of novel anti-influenza drugs and new vaccines. The World Health Organization analyzes a large amount of data relating to the antigenic and genetic characteristics of influenza virus every year, predicts the possibly emerging influenza virus strains, and provides recommendations regarding the antigens to be used to create influenza vaccines for the following influenza season. Based on this recommendation, pharmaceutical and vaccine regulatory agencies develop, produce, and license influenza virus vaccines under a greatly accelerated and highly expensive time frame. Nonetheless, there is a lag behind the evolution of influenza virus strains, and it has not yet been possible to develop a new anti-influenza drug or vaccine fast enough to combat a new virus strain immediately as it emerges.

### Influenza Virus

In some aspects, the present disclosure provides methods for identifying and/or predicting the emergence of drug-resistant influenza viruses. There are two main types of influenza (flu) virus: types A and B. The influenza A and B viruses that routinely spread in people (human influenza viruses) are responsible for seasonal flu epidemics each year. Influenza A viruses can be broken down into sub-types depending on the genes that make up the surface proteins. Over the course of a flu season, different types (A & B) and subtypes (e.g., influenza A) of influenza circulate and cause illness.

There are four types of influenza viruses: A, B, C and D. Human influenza A and B viruses cause seasonal epidemics of disease almost every winter in the United States. The emergence of a new and very different influenza A virus to infect people can cause an influenza pandemic. Influenza type C infections generally cause a mild respiratory illness and are not thought to cause epidemics. Influenza D viruses primarily affect cattle and are not known to infect or cause illness in people. Influenza A viruses are divided into subtypes based on two proteins on the surface of the virus: the hemagglutinin (H) and the neuraminidase (N). There are 18 different hemagglutinin subtypes and 11 different neuraminidase subtypes. (H1 through H18 and N1 through N11 respectively.) Influenza A viruses can be further broken down into different strains. Current subtypes of influenza A viruses found in people are influenza A (H1N1) and influenza A (H3N2) viruses. In the spring of 2009, a new influenza A (H1N1) virus (CDC 2009 H1N1 Flu website) emerged to cause illness in people. This virus was very different from the human influenza A (H1N1) viruses circulating at that time. The new virus caused the first influenza pandemic in more than 40 years. That virus (often called “2009 H1N1”) has now replaced the H1N1 virus that was previously circulating in humans. Herein, “H1N1” refers to any H1N1 virus circulating in humans. Influenza B viruses are not divided into subtypes, but can be further broken down into lineages and strains. Currently circulating influenza B viruses belong to one of two lineages: B/Yamagata and B/Victoria. See, e.g., cdc.gov/flu/about/viruses/types.htm (Centers for Disease Control and Prevention website).

Some methods of the present disclosure comprise evolving and/or culturing a parent strain of influenza viral particles in cell culture comprising human airway (e.g., lung) cells in the presence of an anti-influenza drug. Other methods of the present disclosure comprise culturing a drug-sensitive parent strain of influenza viral particles in cell culture comprising human airway cells in the presence of an anti-influenza drug.

The parent strains of influenza virus (and/or the progeny) may be any one of the four types of influenza viruses, although in preferred embodiments, the parent strain of influenza virus is an influenza type A virus, an influenza type B virus, or an influenza type C virus.

In some embodiments, an influenza A strains is selected from the following subtypes: H1N1, H1N2, H1N3, H1N8, H1N9, H2N2, H2N3, H2N8, H3N1, H3N2, H3N8, H4N2, H4N4, H4N6, H4N8, H5N1, H5N2, H5N3, H5N6, H5N8, H5N9, H6N1, H6N2, H6N4, H6N5, H6N6, H6N8, H7N1, H7N2, H7N3, H7N7, H7N8, H7N9, H8N4, H9N1, H9N2, H9N5, H9N8, H10N3, H10N4, H10N7, H10N8, H10N9, H11N2, H11N6, H11N9, H12N1, H12N3, H12N5, H13N6, H13N8, H14N5, H15N2, H15N8, H16N3, H17N10, and H18N11. In some embodiments, the strain of influenza virus is an influenza A (H1N1) strain. In some embodiments, the strain of influenza virus is an influenza A (H3N2) strain. In some embodiments, the strain of influenza virus is an influenza A (H5N1) strain. Non-limiting examples of particular strains of influenza virus include influenza A/WSN/33 (H1N1), influenza A/Hong Kong/8/68 (H3N2), and influenza A/Avian Influenza (H5N1), influenza A/Netherlands/602/2009 (H1N1), and influenza A/Panama/2007/99 (H3N2).

An influenza virion is roughly spherical and the basic structure includes a lipid bilayer outer membrane, which harbors glycoproteins HA (hemagglutinin) and NA (neuraminidase), the proteins that determine the subtype of influenza virus, and the ion channel M2. Beneath the lipid bilayer is a matrix protein (M1), which forms a shell, giving strength and rigidity to the outer membrane. Within the interior of the virion are viral RNAs, referred to as RNA segments, that code for one or two proteins. Each RNA segment includes RNA joined with several proteins, including B1, PB2, PA, NP. These RNA segments are the genes of influenza virus. The interior of the virion also contains another protein referred to as NEP.

When an influenza virus infects a cell, the individual RNA segments of the influenza virus are replicated in the nucleus. The replicated RNA segments are exported to the cytoplasm, and are incorporated into new viral particles that bud from the cell. If a cell is infected with multiple different influenza virus strains, replicated RNA segments from one virus strain can be incorporated into viral particles with replicated RNA segments from another virus strain to form a reassorted influenza virus. Reassortment refers to influenza viruses containing RNA segments from more than influenza virus strain.

Reassortment of influenza virus in vivo gives rise to new influenza virus strains. These new influenza virus strains can rapidly spread through a naïve population and can lead to an influenza outbreak. A naïve population has never encountered an antigen (e.g., influenza virus antigens) and thus has no immunity against the antigen. Methods of predicting the reassortment of influenza virus can be used to predict new influenza virus strains that can lead to outbreaks. Thus, the present disclosure also provides methods for predicting influenza gene reassortment.

### Drug Sensitivity and Drug Resistance

A drug-sensitive influenza virus is an influenza virus that, when contacted with (exposed to, cultured in the presence of) one or more anti-influenza drug (e.g., cultured in the presence of the drug or otherwise exposed to the drug in vitro or in vivo) no longer enters into host cells, no longer replicates (multiplies) in host cells, no longer releases from host cells, and/or no longer spreads throughout the host—the virus is inhibited. While a particular influenza virus strain may be considered drug-sensitive (e.g. sensitive to oseltamivir), there may be a certain percentage (e.g., less than 30%, less than 20%, or less than 10%) of viral particles among a particular population of influenza viral particles of a particular strain that are not drug sensitive. These viral particles that are not sensitive to the drug—that continue to replicate and/or spread in the presence of the drug—are considered drug resistant. Thus, a drug-resistant influenza virus is an influenza virus that, when contacted with one or more anti-influenza drug (e.g., cultured in the presence of the drug or otherwise exposed to the drug in vitro or in vivo) continues to replicate—viral replication is not inhibited.

An anti-influenza drug is a drug that inhibits (e.g., prevents/inactivates) activity or expression of an influenza viral protein. In some embodiments, an anti-influenza virus drug inhibits influenza virus M1 protein, M2 protein, HA protein, NA protein, or a viral polymerase (e.g., subunit PB1, PA, and/or P3). Non-limiting examples of anti-influenza drugs (drugs that inhibit replication and/or spread of an influenza virus) include oseltamivir (TAMIFLU®), peramivir (RAPIVAB®), zanamivir (RELENZA®), amantadine (SYMMETREL®), rimantadine (FLUMADINE®), and baloxavir marboxil (XOFLUZA®). In some embodiments, the anti-influenza drug is oseltamivir (TAMIFLU®). In some embodiments, the anti-influenza drug is peramivir (RAPIVAB®). In some embodiments, the anti-influenza drug is zanamivir (RELENZA®). In some embodiments, the anti-influenza drug is amantadine (SYMMETREL®). In some embodiments, the anti-influenza drug is rimantadine (FLUMADINE®). In some embodiments, the anti-influenza drug is baloxavir marboxil (XOFLUZA®).

In some embodiments, the anti-influenza drug inhibits the matrix 2 (M2) protein on the surface of the influenza virus. Anti-influenza drugs that inhibit the M2 protein decrease the replication of the influenza viral particle. In some embodiments, the anti-influenza drug that inhibits the M2 protein of the influenza virus is amantadine or rimantadine.

In some embodiments, the anti-influenza drug inhibits the neuraminidase (NA) protein on the surface of the influenza virus. Anti-influenza drugs that inhibit the NA protein decrease the secretion of influenza viral particles and thus inhibit influenza virus spread. In some embodiments, the anti-influenza drug that inhibits the NA protein of the influenza virus is oseltamivir, peramivir, or zanamivir.

More than one drug may be used in the methods described herein. In some embodiments, a cell culture includes 1, 2, 3, 4, 5, 6, 7, 8, 9, or 10 anti-influenza drugs. In some embodiments, a cell culture includes at least 1, at least 2, at least 3, at least 4, at least 5, at least 6, at least 7, at least 8, at least 9, or at least 10 anti-influenza drugs. In some embodiments, a cell culture includes one anti-influenza drug. In some embodiments, a cell culture includes two anti-influenza drugs.

The concentration of anti-influenza drug used herein may vary. In some embodiments, the anti-influenza drug(s) is present in the cell culture at a concentration of 0.5 μM to 10 μM. For example, the anti-influenza drug(s) may be present in the cell culture at a concentration of 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, or 10 3 μM. In some embodiments, the anti-influenza drug(s) is present in the cell culture at a concentration of 0.5-10 μM, 0.5-9 μM, 0.5-8 μM, 0.5-7 μM, 0.5-6 μM, 0.5-5 μM, 0.5-4 μM, 0.5-3 μM, 0.5-2 μM, or 0.5-1 μM.

### Assays for Identifying Drug-Resistant Influenza Virus

Some methods herein comprise (a) evolving a (one or more) parent strain of influenza viral particles in cell culture comprising human airway (e.g., lung cells) in the presence of an anti-influenza drug, and (b) isolating drug-resistant progeny influenza viral particles released from the human airway cells.

“Evolving” an influenza virus comprises, in some embodiments, culturing the influenza virus under conditions that result in the emergence of a viral mutation that confers a survival benefit to the influenza virus. For example, evolving a parent strain of influenza viral particles may comprise culturing human airway cells that comprise a drug-sensitive parent strain of influenza viral particles in cell culture that includes an anti-influenza drug for a period of time sufficient to inhibit viral replication and/or viral spread of a subset of the influenza viral particles, and then culturing human airway cells that comprise progeny of the influenza viral particles in cell culture that comprises the anti-influenza drug. In some embodiments, the methods comprises culturing human airway cells that comprise a drug-sensitive parent strain of influenza viral particles in cell culture that includes an anti-influenza drug for a period of time sufficient to reduce influenza viral titer (e.g., by at least 50%, at least 60%, at least 70%, at least 80%, or at least 90%, relative to baseline), and then culturing human airway cells that comprise progeny of the influenza viral particles in cell culture that comprises the anti-influenza drug.

Culturing refers to maintaining infected airway cells in vitro in conditions that promote growth and proliferation. In some embodiments, culturing includes to changing the media (passaging) in which infected airway cells are maintained. In some embodiments, infected cells are cultured for up to 4 weeks in the presence of an anti-influenza drug. In some embodiments, infected cells are cultured for up to 3 weeks in the presence of an anti-influenza drug. In some embodiments, infected cells are cultured for up to 2 weeks in the presence of an anti-influenza drug. In some embodiments, infected cells are cultured for up to 4 days, 1 week, 1.5 weeks, 2 weeks, 2.5 weeks, 3 weeks, 3.5 weeks, or 4 weeks in the presence of an anti-influenza drug.

In some embodiments, human airway cells comprising a drug-sensitive parent strain of influenza viral particles are cultured in the presence of an anti-influenza drug for a period of time sufficient to inhibit viral replication and/or viral spread (secretion from a host cell, e.g., a human airway cell) of at least 50% of the influenza viral particles. In some embodiments, the drug-sensitive parent strain of influenza viral particles are cultured for a period of time sufficient to inhibit viral replication and/or viral spread of at least 50%, at least 60%, at least 70%, at least 80%, or at least 90% of the influenza viral particles. In some embodiments, human airway cells comprising a drug-sensitive parent strain of influenza viral particles are cultured in the presence of an anti-influenza drug for a period of time sufficient to reduce influenza viral titer by at least 50%, relative to baseline. In some embodiments, human airway cells comprising a drug-sensitive parent strain of influenza viral particles are cultured in the presence of an anti-influenza drug for a period of time sufficient to reduce influenza viral titer by at least 50%, at least 60%, at least 70%, at least 80%, or at least 90%, relative to baseline. In some embodiments, the drug-sensitive parent strain of influenza viral particles is cultured in the presence of an anti-influenza drug (e.g., oseltamivir (TAMIFLU®), peramivir (RAPIVAB®), zanamivir (RELENZA®), amantadine (SYMMETREL®), rimantadine (FLUMADINE®), and baloxavir marboxil (XOFLUZA®)) for a period of time sufficient to inhibit viral replication and/or viral spread of at least 90% of the influenza viral particles.

In some embodiments, human airway cells comprising the parent strain of influenza viral particles are cultured for a period of time sufficient to enable multiple rounds of viral replication. For example, human airway cells comprising the parent strain of influenza viral particles may be cultured for a period of time sufficient to enable at least 2, at least 5, at least 10, at least 20, at least 30, at least 40, or at least 50 rounds of viral replication.

The period of time any population of human airway cells is cultured may depend on the desired result, for example, inhibition of viral replication in a certain percentage of the population, or emergence of a certain percentage of drug-resistant progeny viral particles. In some embodiments, the period of time is at least 12 hours, at least 24 hours, at least 36 hours, at least 48 hours, or at least 60 hours. In some embodiments, the period of time is 12-60 hours, 12-48 hours, 12-36 hours, 12-24 hours, 24-60 hours, 24-48 hours, 24-36 hours, 36-60 hours, 36-48 hours, or 48-60 hours.

Replication of a virus can be determined/monitored by measuring viral titer, for example. Viral titer is a measure of the quantity of virus in a given volume. Non-limiting methods of measuring viral titer include viral plaque assay, quantitative polymerase chain reaction (qPCR) of viral proteins, 50% tissue culture infectious dose assay (TCID50), and focus forming assay. A decreased viral titer is indicative of a decrease in viral replication and thus viral spread. An increased viral titer is indicative of an increase in viral replication and thus viral spread.

In some embodiments, the viral titer is reduced by at least 90% in cells cultured in the presence of anti-influenza drug compared with cells not cultured in the presence of the anti-influenza drug. In some embodiments, the viral titer is reduced by at least 50%. In some embodiments, the viral titer is reduced by at least 75%. In some embodiments, contacting the infected airway cells with the anti-influenza drug reduces viral titer by at least 50%, at least 55%, at least 60%, at least 65%, at least 70%, at least 75%, at least 80%, at least 85%, at least 90%, at least 95%, or at least 99% compared to infected airway cells that are not contacted with the anti-influenza drug.

Human airway cells comprising progeny influenza viral particles, in some embodiments, are cultured in the presence of the anti-influenza drug until the rate of viral replication increases to greater than 50% (the rate of inhibition of viral replication decreases). For example, human airway cells comprising the progeny influenza viral particles, in some embodiments, are cultured in the presence of the anti-influenza drug until the rate of viral replication increases to greater than 60%, greater than 70%, greater than 80% or greater than 90%.

Thus, in some embodiments, methods herein comprise culturing human airway cells that comprise a drug-sensitive strain of influenza viral particles in cell culture comprising the drug until the rate of viral inhibition reaches at least 50% (at least 50% of the viral particles are inhibited), and culturing human airway cells that comprise progeny of the influenza viral particles in cell culture comprising human airway cells in the presence of the anti-influenza drug until the rate of viral replication increases to at least 50%. In some embodiments, human airway cells that comprise the drug-sensitive strain of influenza viral particles are cultured until the rate of viral inhibition reaches at least 60%, at least 70%, at least 80%, or at least 90%. In some embodiments, human airway cells that comprise the progeny of the influenza viral particles are cultured until the rate of viral replication increases to at least 60%, at least 70%, at least 80%, or at least 90%.

Culturing of human airway cells that comprise the progeny influenza viral particles, in some embodiments, comprises passaging (subculturing) human airway cells comprising viral particles of the parent strain and/or of progeny of the parent strain. Passaging refers to the process of renewing the cell culture growth media, e.g., to enable further propagation of the viral particles. In some embodiments, the human airway cells are passaged at least 5, at least 10, at least 15, at least 20, at least 25, or at least 30 times, to produce the drug-resistant progeny influenza viral particles. In some embodiments, the human airway cells are passaged 5-50, 5-40, 5-30, 5-25, 5-20, 5-15, 5-10, 10-50, 10-40, 10-30, 10-25, 10-20, or 20-25 times, to produce the drug-resistant progeny influenza viral particles.

Methods herein, in some embodiments, comprise isolating drug-resistant progeny influenza viral particles (e.g., released from human airway cells). Isolating refers to separating viral particles from the culture (e.g., any components in the culture, such as cells). Isolating may be by any method known or developed in the art, including viral plaque assay formation, trypan blue staining, and magnetic sorting using DynaBeads (ThermoFisher Scientific). For example, the drug-resistant progeny influenza viral particles may isolated from drug-resistant virus pools through viral plaque purification. Other isolation/purifications may be used.

The method, in some embodiments, further comprise sequencing viral RNA obtained from the drug-resistant progeny influenza viral particles to identify a (one or more) drug-resistant strain of influenza virus comprising a mutation in its genome, relative to the parent strain of influenza virus. Any sequencing method may be used. See, e.g., Marston D A et al. BCM Genomics 2013; 14:444; Goya S et al. PLoS One 2018; 13(6): e0199714; and Keller M W et al. Scientific Reports 2018; 8(14408): 1-8, each of which is incorporated herein by reference.

The influenza viral particles herein evolved under the selective pressure of an anti-influenza drug may acquire one or more mutation (e.g., in a viral protein, such as M1 protein, M2 protein, HA protein, NA protein, and/or a viral polymerase (e.g., subunit PB1, PA, and/or P3)) that confers resistance to the anti-influenza drug. The mutation may be any mutation that results in a change in the amino acid sequence of the progeny viral particles, relative to the parent viral particles. Examples of mutations include point mutations (substitutions), insertions, and deletions. The mutation may be any one, or any combination, of the foregoing mutations. In some embodiments, the influenza viral particles acquire at least 2 mutations in an influenza viral protein. In some embodiments, the live infected airway cells comprise at least 3, 4, 5, 6, 7, 8, 9, or 10 mutations in an influenza viral protein.

### Immunogenic Compositions/Vaccines

Also provided herein are methods of developing (producing) a vaccine or other immunogenic composition against the drug-resistant strain of influenza virus. Methods of making influenza virus (flu) vaccines are known, including egg-based flu vaccines, cell-based flu vaccines, and recombinant flu vaccine. See, e.g., Centers for Disease Control and Prevention website (cdc.gov) and the U.S. Food and Drug Vaccine Product Approval Process, each of which is incorporated herein by reference. Non-limiting examples of vaccines that may be developed as provided herein include live-attenuated virus vaccines, inactivated viral vaccines, recombinant viral vaccines, polypeptide vaccines, DNA vaccines, RNA vaccines, and virus-like particles.

The present disclosure provides, in some embodiments, immunogenic compositions for preventing and/or treating influenza (influenza virus infection). These compositions (e.g., pharmaceutical compositions) include at least one influenza virus antigen, or nucleic acid encoding influenza virus antigen, of a variant influenza virus strain identifying using the methods of the present disclosure. Antigens are proteins capable of inducing an immune response (e.g., causing an immune system to produce antibodies against the antigens). An immunogenic fragment induces or is capable of inducing an immune response to influenza. It should be understood that the term “protein” encompasses polypeptides and peptides and the term “antigen” encompasses antigenic fragments.

In some embodiments, an immunogenic composition comprises an influenza virus matrix 2 (M2) antigen variant that comprises an amino acid substitution at position 31, relative to a H1N1 influenza virus M2 antigen, wherein the H1N1 influenza virus M2 antigen comprises the amino acid sequence of SEQ ID NO: 3. In some embodiments, the influenza virus M2 antigen variant further comprises an amino acid substitution at position 34. In some embodiments, the influenza virus M2 antigen variant further comprises an amino acid substitution at position 46. In some embodiments, the influenza virus M2 antigen variant further comprises an amino acid substitution at position 31 and at position 34. In some embodiments, the influenza virus M2 antigen variant further comprises an amino acid substitution at position 31 and at position 46. In some embodiments, the amino acid substitution at position 31 is S31N. In some embodiments, the amino acid substitution at position 34 is G34E. In some embodiments, the amino acid substitution at position 46 is L46P. In some embodiments, the influenza virus M2 antigen variant comprises the amino acid sequence of SEQ ID NO: 1. In some embodiments, the influenza virus M2 antigen variant comprises an amino acid sequence that shares at least 90% identity or at least 95% identity with the amino acid sequence of SEQ ID NO: 1. In some embodiments, the influenza virus M2 antigen variant comprises the amino acid sequence of SEQ ID NO: 2. In some embodiments, the influenza virus M2 antigen variant comprises an amino acid sequence that shares at least 90% identity or at least 95% identity with the amino acid sequence of SEQ ID NO: 2.

Further provided herein, in some embodiments, is a method comprising administering to a subject (e.g., a human subject) the immunogenic composition of any one of the embodiments of the present disclosure in an effective amount to induce in the subject an antigen-specific immune response. In some embodiments, the antigen-specific immune response is a neutralizing antibody response. A “an effective amount” of an influenza immunogenic composition/vaccine is based, at least in part, on the target tissue, target cell type, means of administration, physical characteristics of the polypeptide (e.g., length, three-dimensional structure, and/or amino acid composition), other components of the composition/vaccine, and other determinants, such as age, body weight, height, sex and general health of the subject. Typically, an effective amount of an influenza immunogenic composition/vaccine provides an induced or boosted immune response as a function of antigen production in the cells of the subject.

In some embodiments, an immunogenic composition further comprises a carrier selected from biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences. In some embodiments, an immunogenic composition further comprises an excipient and/or adjuvant.

### Cell Culture

The cell cultures described herein, in some embodiments, include a human small airway-on-chip device. The device, in some embodiments, comprises a polymer chip comprising a membrane that separates (a) an air channel; (b) a microvascular channel; and (c) a membrane, wherein the membrane comprises an epithelium layer exposed to the air channel and an endothelium layer exposed to the microvascular channel. In some embodiments, the air channel is above and/or parallel to the microvascular channel.

In some embodiments, the polymer chip comprises poly(dimethylsiloxane) (PDMS). Other polymers may be used.

In some embodiments, the air channel has a height of 0.5 mm to 2 mm (e.g., 0.5 mm, 1.0 mm, 1.5 mm, or 2 mm). In some embodiments, the air channel has a width of 0.5 mm to 2 mm (e.g., 0.5 mm, 1.0 mm, 1.5 mm, or 2 mm). In some embodiments, the air channel has a diameter of 0.5 mm to 2 mm (e.g., 0.5 mm, 1.0 mm, 1.5 mm, or 2 mm).

In some embodiments, the microvascular channel has a height of 0.1 mm to 2 mm (e.g., 0.1 mm, 0.2 mm, 0.3 mm, 0.4 mm, 0.5 mm, 1 mm, 1.5 mm, or 2 mm). In some embodiments, the microvascular channel has a width of 0.1 mm to 2 mm (e.g., 0.1 mm, 0.2 mm, 0.3 mm, 0.4 mm, 0.5 mm, 1 mm, 1.5 mm, or 2 mm). In some embodiments, the microvascular channel has a diameter of 0.1 mm to 2 mm (e.g., 0.1 mm, 0.2 mm, 0.3 mm, 0.4 mm, 0.5 mm, 1 mm, 1.5 mm, or 2 mm).

In some embodiments, the membrane is a porous membrane. In some embodiments, the porous membrane comprises 0.2 μm to 10 μm pores (e.g., 0.2 am, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, or 10 m.

In some embodiments, membrane has a thickness of 5 am to 15 am (e.g., 5 am, 6 am, 7 m, 8 μm, 9 μm, or 10 μm.

In some embodiments, the membrane is a polyester membrane. Other membrane materials may be used. In some embodiments, the membrane is coated with collagen, for example, type IV collagen. As discussed below, the epithelium layer of the membrane, in some embodiments, comprises primary human lung airway epithelial cells (hLAECs). In some embodiments, the endothelium layer of the membrane comprises primary human lung microvascular endothelial cells (hLMVECs). In some embodiments, the epithelium and/or endothelium layer(s) comprises lung airway epithelial cells and/or lung microvascular endothelial cells that are generated from induced pluripotent stem cells (iPSCs).

The device (e.g., microfluidic device), in some embodiments, has at least one channel (e.g., microchannel) comprising human airway cells, a port at both ends of each microchannel, and one or more pumps for moving a fluid across the at least one microchannel. A microchannel is a channel with a diameter that is less than or equal to 1 millimeter (mm). The ports are sites for the introduction of agents, factors, or cells into the device and for the removal of fluid from the device.

A device of the present disclosure may comprise more than one microchannel. In some embodiments, the device comprises at least two channels (e.g., microchannels). The channels may be configured to mimic a human airway, in which there is an upper microchannel and a lower microchannel separated by a membrane. The membrane may be porous to allow passage of liquids, cells, agents, and/or factors between the upper and the lower channels. In some embodiments, the membrane is coated with extracellular matrix (ECM) proteins to facilitate culture of airway cells. In some embodiments, the ECM proteins are type I collagen, type II collagen, type III collagen, and/or type IV collagen.

Influenza virus primarily infects cells of the airway (e.g., airway epithelium, lung epithelium, airway endothelium, lung endothelium, alveoli). Cells are cultured in the device to mimic the airway of a subject. Airway cells are cells found in the airway of mammals (e.g., humans). The airway refers to the respiratory system, which comprises cells of the pharynx, trachea, and lung (e.g., bronchus, bronchioles, and alveoli). Non-limiting examples of airway cells include epithelial cells, endothelial cells, blood cells, immune cells, cartilaginous cells, and alveoli. The airway comprises epithelial cell and endothelial cell layers, in some embodiments. The epithelial cells are the primary site of influenza infection. Infected epithelial cells signal to endothelial cells to initiate immune cell recruitment. In some embodiments, the cells are epithelial cells (e.g., airway epithelium, lung epithelium). In some embodiments, the cells are endothelial cells (e.g., airway endothelium, lung endothelium). In some embodiments, the cells are epithelial cells and endothelial cells.

Infecting airway cells with an influenza virus refers to contacting airway cells with an influenza virus under conditions that allow infection (e.g., 37° C., 5% CO₂). Infection of airway cells may be confirmed by any method known or developed in the art. Non-limiting methods of confirming influenza virus infection include microscopy to detect the presence of viral particles in the cytoplasm of cells, identification of virial particles budding and being secreted from infected cells, and quantitative PCR (qPCR) using primers that hybridize to influenza virus genes, but not airway cell genes.

### EXAMPLES

### Example 1: Construction of Clinically Relevant In Vitro Model of Influenza Virus Infection on Human Small Airway Chip

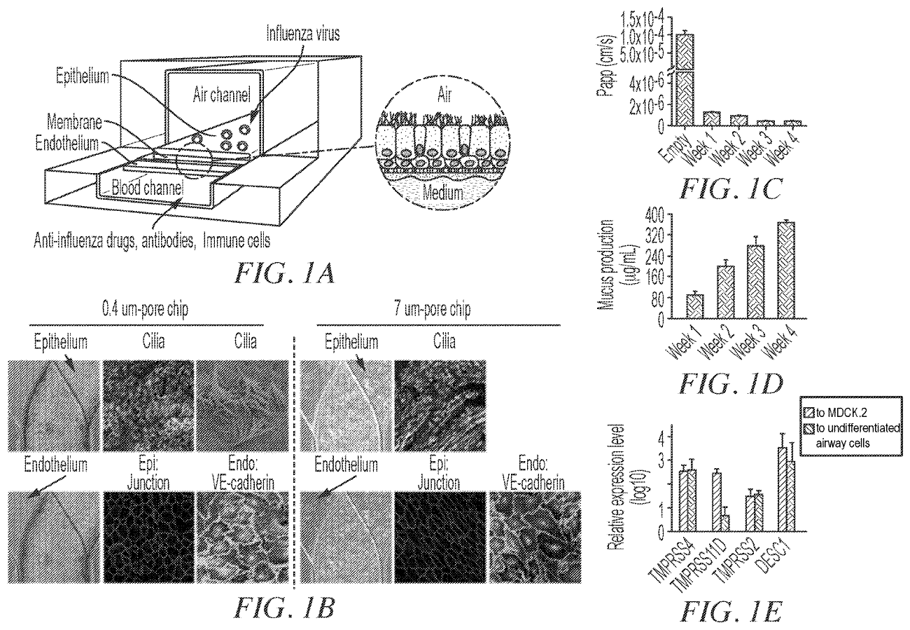

Different types of microfluidic chips made of poly(dimethylsiloxane) (PDMS) containing an upper channel (1 mm high×1 mm wide, similar to the radius of human bronchiole) and a parallel lower microvascular channel (0.2 mm high×1 mm wide) separated by a thin (10 μm), porous, polyester membrane coated on both sides with type IV collaged to construct the human small airway structures (FIG. 1A) (Benam, et al., 2016, Nat. Methods, 13: 151-157; Benam, et al., 2016, Cell Syst, 3: 456-466; Benam, et al., 2017, Methods Mol Biol. 1612: 345-365). The differences among the various types of chips are their different pore sizes on the porous membrane, such as the 0.4 μm and 7 μm-pores. The 7 μm-pore chip allows the immune cells to migrate from the blood channel to the apical channel so that it can be used to study the interaction between immune cells with the influenza infection. After differentiation with the air-liquid interface and differentiation medium flow, the human lung airway-on-chip exhibited tight junctions on the epithelium and endothelium and well-formed cilia (FIG. 1B). This in vitro lung model also exhibited increased barrier function (FIG. 1C) and mucus production (FIG. 1D), compared to controls. Importantly, the in vitro lung model as showed increased expression of a variety of serine proteases, including TMPRSS2, TMPRSS4, TMPRSS11D, and TMPRSS11E (FIG. 1E), which play a role in the activation and propagation of influenza viruses in vivo.

Therefore, different from the previous models, the human lung airway-on-a-chip could effectively recapitulate the structures and functions of in vivo healthy lung bronchioles and sustain them for more than two months in vitro.

### Example 2: Human Small Airway Chip Supports Influenza Virus Infection

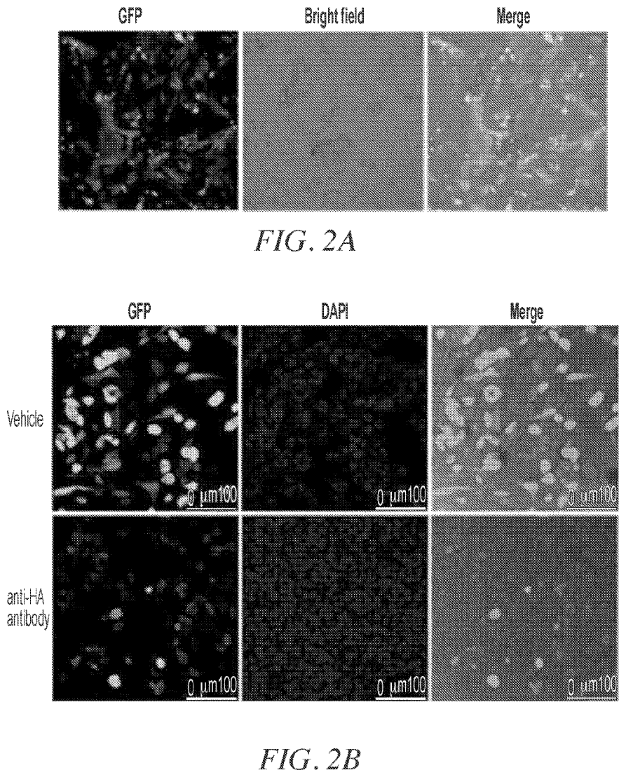

To develop the human small airway-on-a-chip as an influenza virus infection model, the epithelium was inoculated with influenza virus via the air channel, mimicking the infection in vivo (FIG. 1A). The influenza virus was a GFP-labeled PR8 (H1N1) virus (FIGS. 2A-2B), which expresses GFP upon cell infection. Using this H1N1 virus, influenza virus infection was visualized in real time on the small airway chip, suggesting that the human small airway-on-a-chip supports influenza virus infection.

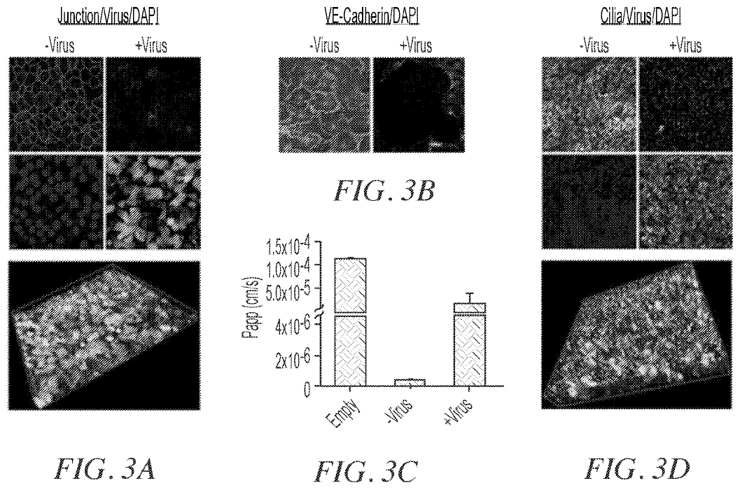

Immunofluorescence confocal microscopic analysis showed that the influenza virus infection damaged the junctions and tissue integrity of epithelium and endothelium (FIG. 3A-3C), as well as the structure of cilia on the epithelium (FIG. 3D).

### Example 3: Viral Replication Kinetics and Cellular Tropism of Influenza Viruses

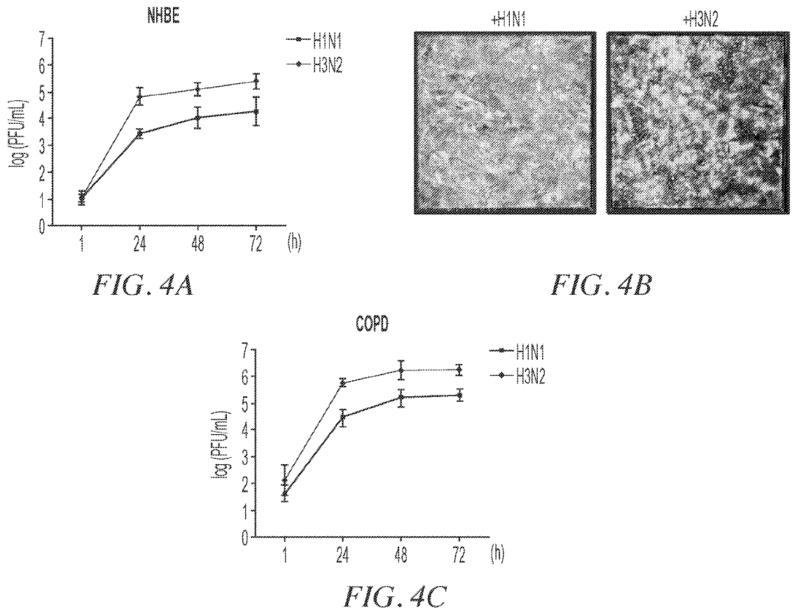

Rapid and direct assessment of the replication capacity of an influenza virus in the upper and conducting airways of humans can provide an important parameter used to assess the zoonotic and pandemic threat posed by emerging influenza viruses. To verify the ability of human small airway chip to assess the viral replication competence, the replication kinetics of influenza A/WSN/33 (H1N1) virus was compared and a human influenza virus strain, e.g., A/Hong Kong/8/68/(H3N2), on chips constructed with human small airway cells from healthy individuals or people with COPD. It was found that the viral titers of both H1N1 and H3N2 viruses increased gradually after inoculation (FIG. 4A); however, H3N2 replicated to significantly higher titers than did H1N1 at each time point of detection (FIG. 4A); in addition, H3N2 infected more cells and caused more cilia loss than H1N1 (FIG. 4B). These suggested that H3N2 has more infectivity and replication competence than H1N1 in human, and can cause more serious damage on human lung airways, consistent with the clinical cases where patients infected with H3N2 showed more severe clinical symptoms than those infected with H1N1. Furthermore, both H1N1 and H3N2 replicated to at least 10-fold higher titers on COPD chip than those on normal chip (FIG. 4C), also consistent with that patients with COPD are more susceptible to infection in clinical, which exacerbates their condition and increases morbidity and mortality.

Cellular tropism could strongly influence influenza severity and pathogenicity [Am J Pathol. 2010 April; 176(4):1614-8]. To show the small airway chip can be used to explore the tropism of influenza viruses, the cellular tropism of three influenza viruses, e.g., H1N1, H3N2, and H5N1 was tested (data not shown). They exhibited different cellular tropism: all three influenza viruses infected goblet cells; a high number of ciliated cells were infected by H1N1 and H3N2 viruses, with none infected by H5N1 virus; a small portion of club cells were infected by all three influenza viruses; and basal cells were infected by H5N1 but not H1N1 or H3N2 (data not shown). Thus, the model can be used to explore the viral tropism of different influenza strains in human and provide information for the prediction of influenza severity and the study of viral pathogenicity.

Collectively, the influenza infection model in the human small airway chip provided results that were consistent with the those observed in clinical studies. Thus, this method can be exploited as an alternative physiologically relevant experimental model for broadening virology research in human physiological environment. In particular, this could include investigation of virus infectivity, replication competence, virulence, and tissue tropism in humans in vitro that could be used to assess the pandemic threat of the emerging influenza viruses, which is a major goal of the World Health Organization (WHO).

### Example 4: Identification of Mutations that Confer Amantadine Resistance

The human small airway-on-a-chip influenza infection model was used to identify a subset of influenza variants that could potentially emerge as a result of evolution during spread from human to human. Knowing these variants would allow one to develop vaccines that can be manufactured in advance and administered to populations as soon as a given variant is identified in the population.

The clinically approved anti-influenza drugs amantadine and oseltamivir were used to identify drug-resistant influenza strains using the human small airway-on-a-chip model. Amantadine targets the M2 protein of influenza viruses, which is an ion channel allowing protons to move through the viral envelope to uncoat viral RNA and thus, it blocks the release of viral RNA into the cytoplasm. Oseltamivir (TAMIFLU®) targets the neuraminidase (NA) protein of influenza virus, inhibiting its enzymatic activity and causing the tethered progeny virus to be unable to escape from the host cell.

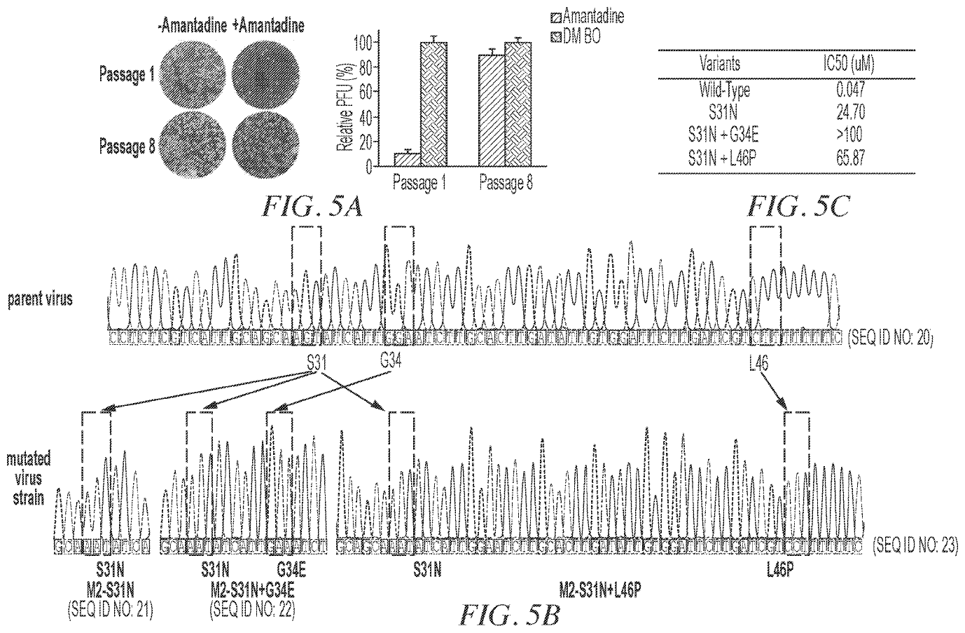

1 μM of amantadine inhibited ˜90% amantadine-sensitive influenza A/WSN/33 strain (H1N1) (FIG. 5A), and thus allowed a low-level viral replication, giving the progeny virus a chance to adapt to the selective pressure. Therefore, 1 μM of amantadine was added to the medium that was flowed through the vascular channel of the airway chip, and a multi-passaging experiment was initiated. Briefly, human small airway chips were infected with amantadine-sensitive influenza A/WSN/33 (MOI=0.1) and treated with 1 μM of amantadine or left untreated for 48 hours (h). Then the apical channels of chips were washed with 50 μl PBS and supernatants containing released viral particles were taken and employed for infection of new human airway chips in the next round of investigation. After each passage, the apical channels of chips were washed with PBS and supernatants were assayed for progeny virus yields by plaque assay. Virus yields of mock-treated cells were arbitrarily set as 100%. This procedure was repeated until viral resistance was induced. The drug-resistant progeny virus strains were isolated from the drug-resistant virus pools through plaque purification, and sequenced to investigate whether any mutation occurred in their genome.

The results show that the inhibition rate of 1 μM of amantadine on influenza virus is ˜90% (FIG. 5A), however, the inhibition rate decreased to ˜10% after 8 passages on chip (FIG. 5A), indicating that the virus pool became resistant to amantadine after 8 passages. After sequencing of the isolated virus strains from the amantadine-resistant virus pool, three mutated virus strains (FIGS. 5B and 5C) were found. The mutations occurred on influenza viral M2 protein that is the target of Amantadine (FIG. 5B). Among them, the single mutation S31N of the M2 protein conferred Amantadine resistance with the IC50 increasing from 47 nM to 24.7 μM (FIG. 5C), which was consistent with the clinical cases wherein a high prevalence of Amantadine resistance due to the substitution of S31 by asparagine (N) has been confirmed in all three circulating subtypes, e.g., H1N1, H3N2, and H1N2. In addition, double mutants with the S31N and either of the G34E or L46P substitutions were observed in the other two Amantadine-resistant strains, respectively (FIG. 5B), which conferred more amantadine resistance with the IC50 increasing from 47 nM to >100 and 65.87 μM, respectively (FIG. 5C). The two double mutants are two new amantadine-resistant virus strains identified in the influenza infection chip. Deeper sequencing might reveal more mutants. These results indicated that our influenza infection chip can be used to evaluate the drug-resistance of current and novel anti-influenza drugs, so that it can be used to not only confirm the drug-resistant virus strains emerging in the clinical setting, but also predict the possibly emerging drug-resistant virus strains.

### Example 5: Identification of Mutations that Confer Oseltamivir Resistance

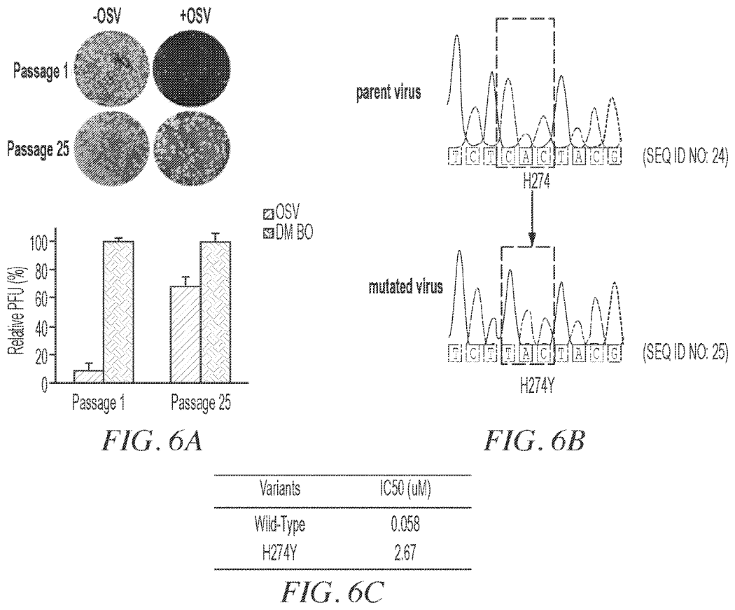

The propensity of oseltamivir to induce viral resistance was also explored (FIGS. 6A-6C). 1 μM of oseltamivir inhibited ˜90% influenza A/WSN/33 strain (H1N1) (FIG. 6A), and thus allowed a low-level viral replication, giving the progeny virus a chance to adapt to the selective pressure. Therefore, 1 μM of oseltamivir was used to conduct the oseltamivir-resistance assay. The results showed that the inhibition rate of 1 μM of oseltamivir on influenza virus is ˜90%, however, the inhibition rate decreased to ˜30% after 25 passages on chip (FIG. 6A), indicating that the virus pool became resistant to oseltamivir after 25 passages. This result also indicated that oseltamivir may have less propensity to induce viral resistance than amantadine. After sequencing of isolated virus strains from the oseltamivir-resistant virus pool, one mutated virus strain was found (FIG. 6B). The mutation occurred on influenza viral NA protein that is the target of oseltamivir. The single mutation H274Y of the NA protein conferred oseltamivir resistance with the IC50 increasing from 58 nM to 2.67 μM (FIG. 6C), which was consistent with the clinical cases wherein the oseltamivir resistance due to the substitution of H274 by Y has also been found. Deeper sequencing might reveal more mutants. These results further confirmed that the influenza infection human small-airway-on-a-chip model can be used to evaluate the drug-resistance of current and novel anti-influenza drugs, so that it can be used to not only confirm the existence of drug-resistant virus strains emerging in patient populations, but also predict the possible virus variant sequences that are responsible for these properties. These variants could represent outstanding targets for proactive vaccine development.

### Example 6: Identification of Reassorted Virus Strains

The ability of the human small airway-on-a-chip model to mimic the influenza virus evolution through gene recombination that causes antigen drift and shift of influenza virus sequences, which is often responsible for the reduction of the efficacy of influenza vaccines in clinical populations was studied. In this proof-of-principle study, the human airway chips were co-infected by the two virus strains, e.g., influenza A/WSN/33 (H1N1) virus (MOI=0.01) and influenza A/Hong Kong/8/68 (H3N2) virus (MOI=0.01), and cultured for 48 h. The progeny virus strains were isolated through plaque purification, and their genomes were sequenced. The sample preparation procedure for sequencing is as follows: The isolated drug-resistant virus strains were cultured in MDCK.2 cells, total RNA was isolated from cells using TRIzol. Then the first strand of cDNA was synthesized using AMV reverse transcriptase (Promega, Madison, Wis., USA) with a random primer and an oligo (dT) primer, according to manufacturer's specifications.

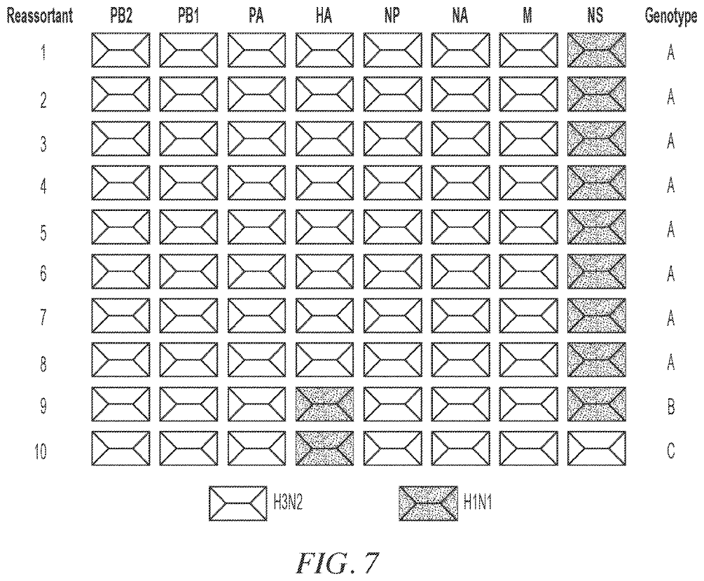

Ten reassortant virus strain variants were detected in the progeny viruses isolated from human airway co-infected by H1N1 and H3N2 viruses (FIG. 7). Based on phylogenetic analyses of the gene segments, the reassortants can be divided into three distinct genotypes (A, B, and C) (FIG. 7). Among the ten reassortants, eight reassortants in genotype A are new H3N2 reassortants containing the NS gene segment from influenza A/WSN/33 (H1N1) virus and the rest of the gene segments from influenza A/Hong Kong/8/68 (H3N2) virus. One reassortant in genotype B is H1N2 reassortant containing the HA and NS genes from influenza A/WSN/33 (H1N1) virus and the rest of the gene segments from influenza A/Hong Kong/8/68 (H3N2) virus. One reassortant in genotype C is H1N2 reassortant containing the HA from influenza A/WSN/33 (H1N1) virus and the rest of the gene segments from influenza A/Hong Kong/8/68 (H3N2) virus.

These results suggest that the influenza human small airway-on-a-chip can be used to mimic the gene recombination of influenza viruses and predict potentially novel emerging reassortants that might cause pandemics. Hundreds of influenza viruses have been identified have been identified in the past. Gene recombination and reassortant between these hundreds of influenza viruses can be explored extensively in the human airway chip so that we can predict the potential emerging reassortants that have increased virulence, and hence may cause influenza pandemics. Therefore, the model can provide substantial information for influenza vaccine design.

### Materials and Methods

PCR was carried out using the Phusion Hot Start Flex 2× Master Mix (New England BioLab, USA) with 30 μl of a reaction mixture containing primers specific for different influenza A/WSN/33 (H1N1) gene segments. The PCR conditions were 1 cycle at 98° C. for 2 min, followed by 30 cycles at 98° C. for 15 sec, 55° C. for 30 sec, 72° C. (30 sec/kb), and finally 1 cycle at 72° C. for 5 min. The resulting PCR products were gene sequenced. The viral genome sequencing could be also done through next generation sequencing service provided by many sequencing companies.

```
SEQUENCES
Amino Acid Sequences
>SEQ ID NO: 1 amino acid sequence of M2 protein (H1N1 strain)
with S31N and G34E mutations
MSLLTEVETPIRNEWGCRCNDSSDPLVIAANIIEILHLILWILDRLFFKCIYRRFKYGLKRG
PSTEGVPESMREEYRKEQQNAVDVDDGHFVNIELE
>SEQ ID NO: 2 amino acid sequence of M2 protein (H1N1 strain)
with S31N and L46P
mutations
MSLLTEVETPIRNEWGCRCNDSSDPLVIAANIIGILHLILWILDRPFFKCIYRRFKYGLKRG
PSTEGVPESMREEYRKEQQNAVDVDDGHFVNIELE
>SEQ ID NO: 3 amino acid sequence of M2 protein (H1N1 strain)
MSLLTEVETPIRNEWGCRCNDSSDPLVIAASIIGILHLILWILDRLFFKCIYRRFKYGLKRG
PSTEGVPESMREEYRKEQQNAVDVDDGHFVNIELE
Nucleic Acid Reference Sequences
PB2 of influenza A/Hong Kong/8/68 (H3N2) virus (SEQ ID NO: 4):
      1 atggaaagaa taaaagaact acggaatctg atgtcgcagt ctcgcactcg cgagatacta
    61 acaaaaacca cagttgacca tatggccata attaagaagt atacatcagg gagacaggaa
  121 aagaacccgt cacttaggat gaaatggatg atggcaatga aatatccaat tacagctgac
  181 aagaggataa cagaaatggt tcctgagaga aatgagcaag gacaaactct atggagcaaa
  241 atgagtgatg ccggatcaga tcgagtgatg gtatcaccct tggcagtgac atggtggaat
  301 agaaatggac caatgacaag tacggttcat tatccaaaag tctacaagac ttattttgag
  361 aaagtcgaaa ggttaaaaca tggaaccttt ggccctgtcc attttagaaa ccaagtcaaa
  421 atacgccgaa gagttgacat aaaccctggt catgcagacc tcagtgccaa ggaggcacaa
  481 gatgtaatca tggaagttgt tttccccaat gaagtggggg ccagaatact aacgtcggaa
  541 tcacaattaa caataaccaa agagaaaaaa gaagaactcc aagattgcaa aatttctcct
  601 ttgatggttg catacatgtt agagagagaa cttgtccgaa aaacgagatt tctcccagtt
  661 gctggtggaa caagcagtgt atacatcgaa gtgttacact tgactcaagg aacgtgttgg
  721 gaacagatgt acactccagg tggagaagtg aggaatgatg atgttgatca aagtctaatt
  781 attgcagcca ggaacatagt gagaagagca gcagtatcag cagatccact agcatcttta
  841 ttggagatgt gccacagcac acagattggc gggacaagga tggtggacat tcttaggcag
  901 aacccaacgg aagaacaagc tgtggatata tgcaaagctg caatgggact gagaatcagc
  961 tcgtccttca gttttggcgg attcacattt aagagaacaa gcgggtcatc aatcaagaga
1021 gaggaagaat tgcttacggg caatctccaa acattaaaaa taagggtgca tgaggggtac
1081 gaggaattca caatggtggg gaaaagggca acagctatac tcagaaaagc aaccaggaga
1141 ttggttcagc tgatagtgag tggaagagac gaacagtcag tagccgaagc aataattgta
1201 gccatggtgt tttcacaaga agattgcatg ataaaagcag ttagaggtga tctgaatttc
1261 gttaacaggg caaatcagcg attgaatccc atgcatcaac ttttaaggca ttttcagaaa
1321 gatgcgaaag tgctttttca aaattgggga attgaacata tcgacaatgt aatggggatg
1381 attggagtat taccagacat gactccaagc acagagatgt caatgagagg gataagagtc
1441 agcaaaatgg gcgtggatga atactccagc acagagaggg ttgtggtgag cattgaccgg
1501 tttttgagag ttcgagacca acgaggaaat gtattactat ctcctgagga ggtcagtgaa
1561 acacagggga cagagaaact gacaataact tactcatcgt caatgatgtg ggagattaat
1621 ggccctgagt cagtgttggt caatacctat cagtggatca tcagaaactg ggaaactgtc
1681 aaaattcaat ggtctcagaa tcctacaatg ttatacaaca aaatggaatt tgagccattt
1741 cagtctttag ttcctaaggc cattagaggc caatacagtg gatttgttag gactctattc
1801 caacaaatga gggatgtact tgggacattt gataccaccc agataataaa gcttctcccc
1861 tttgcagccg ccccaccaaa gcaaagtagg atgcagttct cttcattgac tgtgaatgtg
1921 aggggatcag ggatgagaat acttgtaagg ggcaattctc ctgtattcaa ctacaacaag
1981 acaacgaaaa gactaacaat tctcggaaaa gatgctggca ctttaattga agacccagat
2041 gaaggtacat ccggagtgga gtcagctgtt ctgagagggt tcctcattct gggtaaggaa
2101 gatagaagat atggaccagc attaagcatc aatgaactga gtaaccttgc aaaaggagaa
2161 aaggctaatg tactaattgg gcaaggagac gtggtgttgg taatgaaacg aaaacgggac
2221 tctagcatac ttactgacag ccagacagcg accaaaagaa ttcggatggc catcaattaa
PB1 of influenza A/Hong Kong/8/68 (H3N2) virus (SEQ ID NO: 5):
      1 atggatgtca atccgacttt acttttcttg aaagttccag cgcaaaatgc cataagcacc
    61 acattccctt atactggaga tcctccatac agccatggaa caggaacagg atacaccatg
  121 gacacagtca acagaacaca tcaatattca gaaaaaggga agtggacaac aaacacggaa
  181 actggagcgc cccaacttaa cccaattgat ggaccactac ctgaggataa tgagccaagt
  241 ggatatgcac aaacagactg tgtcctggaa gcaatggctt tccttgaaga atcccaccca
  301 gggatctttg aaaactcgtg tcttgaaacg atggaagttg ttcaacaaac aagggtggac
  361 agactgaccc aaggtcgtca gacctatgat tggacattaa acagaaatca accggccgca
  421 actgcattag ccaacactat agaagtcttc agatcgaatg gtctaacagc taatgagtcg
  481 ggaaggctaa tagatttcct caaagatgtg atggaatcaa tggataaaga ggaaatggag
  541 ataacaacac acttccaaag aaaaagaaga gtaagagaca acatgaccaa gaaaatggtc
  601 acacaaagaa caataggaaa gaagaagcag agagtgaaca agagaagcta tctaataaga
  661 gcattaacat tgaacacaat gaccaaagat gcagaaagag gtaaattaaa gagaagagct
  721 attgcaacac ccgggatgca aatcagaggg ttcgtgtact ttgttgaaac tctagctagg
  781 agcatttgtg agaagcttga acagtctgga cttccagttg gaggtaatga aaagaaggcc
  841 aaactggcaa atgttgtgag aaagatgatg actaattcac aagacacaga gctttctttc
  901 acaattactg gagacaatac taaatggaat gaaaatcaaa atcctcgaat gttcctggcg
  961 atgattacat atatcacaaa aaatcaacct gaatggttca gaaacgttct gagcatcgca
1021 cccataatgt tctcaaacaa aatggcgaga ctagggaaag gatacatgtt cgaaagtaag
1081 agcatgaagc tccgaacaca aataccagca gaaatgctag caagcattga cctaaagtat
1141 ttcaatgaat caacaagaaa gaaaattgag aaaataaggc ctcttctaat agatggcaca
1201 gcttcattga gtcctggaat gatgatgggc atgttcaaca tgctaagtac ggttttagga
1261 gtctcaatcc tgaatcttgg gcaaaagaga tacaccaaaa caacatactg gtgggatgga
1321 ctccaatcct ctgatgattt tgctctcata gtgaatgcac caaatcatga gggaatacaa
1381 gcaggagtgg atagattcta cagaacctgc aagttagtcg gaatcaatat gagcaagaag
1441 aagtcctata taaataggac aggaacattt gaattcacaa gctttttcta tcgctatgga
1501 tttgtagcca attttagcat ggagctgccc agttttggag tgtctgggat taatgagtca
1561 gctgatatga gcattggagt aacagtgata aagaacaaca tgataaacaa tgaccttgga
1621 ccagcaacag cccagatggc tcttcaactg ttcatcaagg actacagata tacataccgg
1681 tgccacagag gagacacaca aattcagacg aggagatcat tcgagctaaa gaagctgtgg
1741 gagcaaaccc gctcaaaggc aggactattg gtttcagatg gaggaccaaa cttatacaat
1801 atccggaatc ttcacatccc ggaagtctgc ttaaagtggg agctaatgga tgaggactat
1861 cagggaagac tttgtaatcc cctgaatcca tttgtcagcc ataaggagat tgagtctgta
1921 aacaatgctg tggtaatgcc agctcatggt ccagccaaga gcatggaata tgacgctgtt
1981 gcaactacac actcctggat tcctaagagg aaccgctcta tcctcaacac aagccaaagg
2041 ggaattcttg aggatgaaca gatgtatcag aagtgctgca acctgttcga gaaatttttc
2101 cccagtagtt catacaggag accggttgga atttccagca tggtggaggc catggtgtct
2161 agggcccgga ttgatgccag aatagacttc gagtctggac ggattaagaa agaagagttc
2221 gccgagatca tgaagatctg ttccaccatt gaagagctca gacggcaaaa atag
PA of influenza A/Hong Kong/8/68 (H3N2) virus (SEQ ID NO: 6):
      1 atggaagatt ttgtacgaca atgctttaat ccgatgattg tcgaacttgc ggaaaaggca
    61 atgaaagagt atggagagga tcttaaaatc gaaacaaaca aatttgcagc aatatgcact
  121 cacttggaag tatgcttcat gtattcagat tttcatttca tcaatgagca aggcgagtca
  181 atagtggtag aacttgatga tccaaatgca cttttgaagc acagatttga aataatagag
  241 ggaagagacc gcacaatggc ctggacagta gtaaacagta tttgcaacac cacaggagct
  301 gagaaaccga agtttctgcc agatttgtat gattacaagg agaatagatt catcgagatt
  361 ggagtgacaa ggagagaagt ccacatatac taccttgaaa aggccaataa aattaaatct
  421 gagaatacac acatccacat tttctcattc actggggaag aaatggccac aaaggccgac
  481 tacactctcg atgaggaaag cagggctagg atcaaaacca gactattcac cataagacaa
  541 gagatggcca acagaggcct ctgggattcc tttcgtcagt ccgaaagagg cgaagaaaca
  601 attgaagaaa gatttgaaat cacagggaca atgcgcaggc ttgccgacca aagtctcccg
  661 ccgaacttct cctgccttga gaattttaga gcctatgtgg atggattcga accgaacggc
  721 tacattgagg gcaagctttc tcaaatgtcc aaagaagtga atgcaaaaat tgaacctttt
  781 ctgaaaacaa caccaagacc aattagactt ccggatgggc ctccttgttt tcagcggtcc
  841 aaattccttc tgatggatgc tttaaagtta agcattgagg atccaagtca cgagggggag
  901 ggaataccac tatatgatgc gatcaaatgc atgagaacat tttttggatg gaaagaaccc
  961 tatattgtta aaccacacga aaaggggata aatccaaatt atctgctgtc atggaagcaa
1021 gtactggcag aactgcagga cattgaaaat gaggagaaaa ttccaagaac taaaaacatg
1081 aagaaaacga gtcagctaaa gtgggcactt ggtgagaaca tggcaccaga gaaggtagac
1141 tttgacaact gtagagacgt aagcgatttg aagcaatatg atagtgacga acctgaatta
1201 aggtcacttt caagctggat ccagaatgag ttcaacaagg catgcgagct gaccgattca
1261 acttggatag agctcgatga gattggagaa gacgtggctc caattgaata cattgcaagc
1321 atgagaagga attacttcac agcagaggtg tcccattgca gagccacaga atatataatg
1381 aagggggtat acattaatac tgccttgctt aatgcatcct gtgcagcaat ggacgatttc
1441 caactaattc ccatgataag caagtgtaga actaaagagg gaaggcgaaa gaccaattta
1501 tatggcttca tcataaaagg aagatctcac ttaaggaatg acaccgacgt ggtaaacttt
1561 gtgagcatgg agttttctct cactgacccg agacttgagc cacacaaatg ggagaaatac
1621 tgtgtccttg agataggaga tatgctacta agaagtgcta taggccagat gtcaaggcct
1681 atgttcttgt atgtgagaac aaatggaaca tcaaagatta aaatgaaatg gggaatggag
1741 atgaggcgtt gcctccttca gtcactccaa caaatcgaga gtatgattga agcagagtca
1801 tctgtcaaag agaaagacat gaccaaagag ttttttgaga ataaatcaga aacatggccc
1861 attggggagt cccccaaggg agtggaagat ggttccattg ggaaggtctg caggacttta
1921 ttggccaagt cggtattcaa tagcctgtat gcatccccgc aattggaagg gttttcagct
1981 gagtcaagaa aactgcttct tgtcgttcag gctcttaagg acaatcttga acctggaacc
2041 tttgatcttg aggggctata tgaagcaatt gaggagtgcc tgattaatga tccctgggtt
2101 ttgcttaatg cgtcgtggtt caactccttc ctaacacatg cattaagata g
HA of influenza A/Hong Kong/8/68 (H3N2) virus (SEQ ID NO: 7):
      1 atgaagacca tcattgcttt gagctacatt ttctgtctgg ctctcggcca agaccttcca
    61 ggaaatgaca acagcacagc aacgctgtgc ctgggacatc atgcggtgcc aaacggaaca
  121 ctagtgaaaa caatcacaga tgatcagatt gaagtgacta atgctactga gctagttcag
  181 agctcctcaa cggggaaaat atgcaacaat cctcatcgaa tccttgatgg aatagactgc
  241 acactgatag atgctctatt gggggaccct cattgtgatg tttttcaaaa tgagacatgg
  301 gaccttttcg ttgaacgcag caaagctttc agcaactgtt acccttatga tgtgccagat
  361 tatgcctccc ttaggtcact agttgcctcg tcaggcactc tggagtttat cactgagggt
  421 ttcacttgga ctggggtcac tcagaatggg ggaagcaatg cttgcaaaag gggacctggt
  481 agcggttttt tcagtagact gaactggttg accaaatcag gaagcacata tccagtgctg
  541 aacgtgacta tgccaaacaa tgacaatttt gacaaactat acatttgggg ggttcaccac
  601 ccgagcacga accaagaaca aaccagcctg tatgttcaag catcagggag agtcacagtc
  661 tctaccagaa gaagccagca aactataatc ccgaatatcg ggtccagacc ctgggtaagg
  721 ggtctgtcta gtagaataag catctattgg acaatagtta agccgggaga cgtactggta
  781 attaatagta atgggaacct aatcgctcct cggggttatt tcaaaatgcg cactgggaaa
  841 agctcaataa tgaggtcaga tgcacctatt gatacctgta tttctgaatg catcactcca
  901 aatggaagca ttcccaatga caagcccttt caaaacgtaa acaagatcac atatggagca
  961 tgccccaagt atgttaagca aaacaccctg aagttggcaa cagggatgcg gaatgtacca
1021 gagaaacaaa ctagaggcct attcggcgca atagcaggtt tcatagaaaa tggttgggag
1081 ggaatgatag acggttggta cggtttcagg catcaaaatt ctgagggcac aggacaagca
1141 gcagatctta aaagcactca agcagccatc gaccaaatca atgggaaatt gaacagggta
1201 atcgagaaga cgaacgagaa attccatcaa atcgaaaagg aattctcaga agtagaaggg
1261 agaattcagg acctcgagaa atacgttgaa gacactaaaa tagatctctg gtcttacaat
1321 gcggagcttc ttgtcgctct ggagaatcaa catacaattg acctgactga ctcggaaatg
1381 aacaagctgt ttgaaaaaac aaggaggcaa ctgagggaaa atgctgaaga catgggcaat
1441 ggttgcttca aaatatacca caaatgtgac aacgcttgca tagagtcaat cagaaatggg
1501 acttatgacc atgatgtata cagagacgaa gcattaaaca accggtttca gatcaaaggt
1561 gttgaactga agtctggata caaagactgg atcctgtgga tttcctttgc catatcatgc
1621 tttttgcttt gtgttgtttt gctggggttc atcatgtggg cctgccagag aggcaacatt
1681 aggtgcaaca tttgcatttg a
NP of influenza A/Hong Kong/8/68 (H3N2) virus (SEQ ID NO: 8):
      1 atggcgtccc aaggcaccaa acggtcttat gaacagatgg aaactgatgg ggaacgccag
    61 aatgcaactg agatcagagc atccgtcggg aagatgattg atggaattgg acgattctac
  121 atccaaatgt gcactgaact taaactcagt gattatgagg ggcgactgat ccagaacagc
  181 ttaacaatag agagaatggt gctctctgct tttgacgaaa gaaggaataa atatctggaa
  241 gaacatccca gcgcggggaa ggatcctaag aaaactggag gacccatata caagagagta
  301 gatggaaagt ggatgaggga actcgtcctt tatgacaaag aagaaataag gcgaatctgg
  361 cgccaagcca ataatggtga tgatgcaaca gctggtctga ctcacatgat gatctggcat
  421 tccaatttga atgatacaac ataccagagg acaagagctc ttgttcgcac cggcatggat
  481 cccaggatgt gctctctgat gcagggttcg actctcccta gaaggtctgg agctgcaggc
  541 gctgcagtca aaggagttgg gacaatggtg atggagttga taaggatgat caaacgtggg
  601 atcaatgatc ggaacttctg gagaggtgaa aatggacgaa aaacaaggag tgcttacgag
  661 agaatgtgca acattctcaa aggaaaattt caaacagctg cacaaagggc aatgatggat
  721 caagtgagag aaagtcggaa cccaggaaat gctgagatcg aagatctcat ctttctggca
  781 cggtctgcac tcatattgag agggtcagtt gctcacaaat cttgtctgcc cgcctgtgtg
  841 tatggacctg ccgtagccag tggctacgac ttcgaaaaag agggatactc tttagtggga
  901 atagaccctt tcaaactgct tcaaaacagc caagtataca gcctaatcag accgaacgag
  961 aatccagcac acaagagtca gctggtgtgg atggcatgca attctgctgc atttgaagat
1021 ctaagagtat taagcttcat cagagggacc aaagtatccc caagggggaa actttccact
1081 agaggagtac aaattgcttc aaatgaaaac atggatgcta tggaatcaag tactcttgaa
1141 ctgagaagca ggtactgggc cataagaacc agaagtggag gaaacactaa tcaacagagg
1201 gcctctgcag gtcaaatcag tgtgcaacct gcattttctg tgcaaagaaa cctcccattt
1261 gacaaaccaa ccatcatggc agcattcact gggaatacag agggaagaac atcagacatg
1321 agggcagaaa ttataaggat gatggaaggt gcaaaaccag aagaaatgtc cttccagggg
1381 cggggagtct tcgagctctc ggacgaaaag gcagcgaacc cgatcgtgcc ctcttttgac
1441 atgagtaatg aaggatctta tttcttcgga gacaatgcag aggagtacga caattaa
NA of influenza A/Hong Kong/8/68 (H3N2) virus (SEQ ID NO: 9):
      1 atgaatccaa atcaaaagat aataacaatt ggctctgtct ctctcaccat tgcaacagta
    61 tgcttcctca tgcagattgc catcctggta actactgtaa cattgcattt taagcaatat
  121 gagtgcgact cccccgcgag caaccaagta atgccgtgtg aaccaataat aatagaaagg
  181 aacataacag agatagtgta tttgaataac accaccatag agaaagagat atgccccaaa
  241 gtagtggaat acagaaattg gtcaaagccg caatgtcaaa ttacaggatt tgcacctttt
  301 tctaaggaca attcaatccg gctttctgct ggtggggaca tttgggtgac gagagaacct
  361 tatgtgtcat gcgatcatgg caagtgttat caatttgcac tcgggcaggg gaccacacta
  421 gacaacaaac attcaaatga cacaatacat gatagaatcc ctcatcgaac cctattaatg
  481 aatgagttgg gtgttccatt tcatttagga accaggcaag tgtgtatagc atggtccagc
  541 tcaagttgtc acgatggaaa agcatggctg catgtttgta tcactgggga tgacaaaaat
  601 gcaactgcta gcttcattta tgacgggagg cttgtggaca gtattggttc atggtctcaa
  661 aatatcctca gaacccagga gtcggaatgc gtttgtatca atgggacttg cacagtagta
  721 atgactgatg gaagtgcttc aggaagagcc gatactagaa tactattcat tgaagagggg
  781 aaaattgtcc atattagccc attgtcagga agtgctcagc atgtagaaga gtgttcctgt
  841 tatcctagat atcctggcgt cagatgtatc tgcagagaca actggaaagg ctctaatagg
  901 cccgtcgtag acataaatat ggaagattat agcattgatt ccagttatgt gtgctcaggg
  961 cttgttggcg acacacctag aaacgacgac agatctagca atagcaattg caggaatcct
1021 aacaatgaga gagggaatca aggagtgaaa ggctgggcct ttgacaatgg agatgacgtg
1081 tggatgggaa gaacgatcag caaggattta cgctcaggtt atgaaacttt caaagtcatt
1141 ggtggttggt ccacacctaa ttccaaatcg cagatcaata gacaagtcat agttgacagc
1201 gataatcggt caggttactc tggtattttc tctgttgagg gcaaaagctg catcaatagg
1261 tgcttttatg tggagttgat aaggggaagg aaacaggaga ctagagtgtg gtggacctca
1321 aacagtattg ttgtgttttg tggcacttca ggtacctatg gaacaggctc atggcctgat
1381 ggggcgaaca tcaatttcat gcctatataa
M of influenza A/Hong Kong/8/68 (H3N2) virus (SEQ ID NO: 10):
      1 atgagccttc taaccgaggt cgaaacgtac gttctctcta tcgtcccgtc aggccccctc
    61 aaagccgaga tcgcacagag acttgaagat gtctttgctg ggaagaacac agatcttgag
  121 gctctcatgg aatggctaaa gacaagacca atcctgtcac ctctgactaa ggggattttg
  181 ggatttgtat tcacgctcac cgtgcccagt gagcgaggac tgcagcgtag acgctttgtc
  241 caaaatgccc tcaatgggaa tggggatcca aataacatgg acagagcagt taaactgtat
  301 agaaaactta agagggagat aacattccat ggggccaaag aaatagcact cagttattct
  361 gctggtgcac ttgccagttg catgggcctc atatacaaca ggatgggggc tgtgaccact
  421 gaagtggcct ttggcctggt atgtgcaacc tgtgaacaga ttgctgactc ccagcatagg
  481 tctcataggc aaatggtgac aacaaccaat ccactaataa gacatgagaa cagaatggtt
  541 ctggccagca ctacagctaa ggctatggag caaatggctg gatcgagtga gcaggcagca
  601 gaggccatgg aggttgctag tcaggccagg caaatggtgc aggcaatgag agccattggg
  661 actcatccta gctccagtgc tggtctaaaa gatgatcttc ttgaaaattt gcaggcctat
  721 cagaaacgaa tgggggtgca gatgcaacga ttcaagtgac cctcttgttg ttgctgcgag
  781 tatcatcggg atcttgcact tgatattgtg gattcttgat cgtctttttt tcaaatgcat
  841 ttatcgattc tttgaacacg gtctgaaaag agggccttct acggaaggag tacctgagtc
  901 tatgagggaa gaatatcgaa aggaacagca gagtgctgtg gatgctgacg atagtcattt
  961 tgtcagcata gagctggagt aa
NS of influenza A/Hong Kong/8/68 (H3N2) virus (SEQ ID NO: 11):
      1 atggattcta acactgtgtc aagttttcag gtagattgct tcctttggca tgtccgaaaa
    61 caagttgtag accaagaact aggtgatgcc ccattccttg atcggcttcg ccgagatcag
  121 aagtccctaa ggggaagagg cagcactctc ggtctaaaca tcgaagcagc cacccgtgtt
  181 ggaaagcaga tagtagagag gattctgaag gaagaatccg atgaggcact taaaatgacc
  241 atggcctccg cacctgcttc gcgataccta actgacatga ctattgagga attgtcaagg
  301 gactggttca tgctaatgcc caagcagaaa gtggaaggac ctctttgcat cagaatagac
  361 caggcaatca tggataagaa catcatgttg aaagcgaatt tcagtgtgat ttttgaccgg
  421 ctagagaccc taatattact aagggctttc accgaagagg gagcaattgt tggcgaaatc
  481 tcaccattgc cttctcttcc aggacatact attgaggatg tcaaaaatgc aattggggtc
  541 ctcatcggag gacttgaatg gaatgataac acagttcgag tctctaaaac tctacagaga
  601 ttcgcttggg gaagcagtaa tgagaatggg agacctccac tcactccaaa acagaaacgg
  661 aaaatggcga gaacagttag gtcaaaagtt cgaagagata agatggctga ttgaagaagt
  721 gagacacaga ttgaagacaa cagagaatag ttttgagcaa ataacattta tgcaagcctt
  781 acagctacta tttgaagtgg aacaggagat aagaactttc tcgtttcagc ttatttaa
HA of influenza A/W5N/33 (H1N1) virus (SEQ ID NO: 12):
      1 atgaaggctt ttgtactagt cctgttatat gcatttgtag ctacagatgc agacacaata
    61 tgtataggct accatgcgaa caactcaacc gacactgttg acacaatatt cgagaagaat
  121 gtggcagtga cacattctgt taacctgctc gaagacagac acaacgggaa actatgtaaa
  181 ttaaaaggaa tagccccact acaattgggg aaatgtaaca tcaccggatg gctcttggga
  241 aatccagaat gcgactcact gcttccagcg agatcatggt cctacattgt agaaacacca
  301 aactctgaga atggagcatg ttatccagga gatttcatcg actatgagga actgagggag
  361 caattgagct cagtatcatc attagaaaga ttcgaaatat ttcccaagga aagttcatgg
  421 cccaaccaca cattcaacgg agtaacagta tcatgctccc ataggggaaa aagcagtttt
  481 tacagaaatt tgctatggct gacgaagaag ggggattcat acccaaagct gaccaattcc
  541 tatgtgaaca ataaagggaa agaagtcctt gtactatggg gtgttcatca cccgtctagc
  601 agtgatgagc aacagagtct ctatagtaat ggaaatgctt atgtctctgt agcgtcttca
  661 aattataaca ggagattcac cccggaaata gctgcaaggc ccaaagtaaa agatcaacat
  721 gggaggatga actattactg gaccttgcta gaacccggag acacaataat atttgaggca
  781 actggtaatc taatagcacc atggtatgct ttcgcactga gtagagggtt tgagtccggc
  841 atcatcacct caaacgcgtc aatgcatgag tgtaacacga agtgtcaaac accccaggga
  901 tctataaaca gcaatctccc tttccagaat atacacccag tcacaatagg agagtgccca
  961 aaatatgtca ggagtaccaa attgaggatg gttacaggac taagaaacat cccatccatt
1021 caatacagag gtctatttgg agccattgct ggttttattg aggggggatg gactggaatg
1081 atagatggat ggtatggtta tcatcatcag aatgaacagg gatcaggcta tgcagcggat
1141 caaaaaagca cacagaatgc cattaacagg attacaaaca aggtgaactc tgttatcgag
1201 aaaatgaaca ctcaattcac agctgtgggt aaagaattca acaacttaga aaaaaggatg
1261 gaaaatttaa ataaaaaagt tgatgatggg tttctggaca tttggacata taatgcagaa
1321 ttgttagttc tactggaaaa tgaaagaact ttggatttcc atgacttaaa tgtgaagaat
1381 ctgtacgaga aagtaaaaag ccaattaaag aataatgcca aagaaatcgg aaatgggtgt
1441 tttgagttct accacaagtg tgacaatgaa tgcatggaaa gtgtaagaaa tgggacttat
1501 gattatccaa aatattcaga agaatcaaag ttgaacaggg aaaagataga tggagtgaaa
1561 ttggaatcaa tgggggtgta tcagattctg gcgatctact caactgtcgc cagttcactg
1621 gtgcttttgg tctccctggg ggcaatcagt ttctggatgt gttctaatgg gtctttgcag
1681 tgcagaatat gcatctga
M of influenza A/WSN/33 (H1N1) virus (SEQ ID NO: 13):
      1 atgagtcttc taaccgaggt cgaaacgtac gttctctcta tcgtcccgtc aggccccctc
    61 aaagccgaga tcgcacagag acttgaagat gtctttgcag ggaagaacac cgatcttgag
  121 gttctcatgg aatggctaaa gacaagacca atcctgtcac ctctgactaa ggggatttta
  181 ggatttgtgt tcacgctcac cgtgcccagt gagcggggac tgcagcgtag acgctttgtc
  241 caaaatgctc ttaatgggaa cgaagatcca aataacatgg acaaagcagt taaactgtgt
  301 aggaagctta agagggagat aacattccat ggggccaaag aaatagcact cagttattct
  361 gctggtgcac ttgccagttg tatgggcctc atatacaaca ggataggggc tgtgaccact
  421 gaagtggcat ttggcctggt atgcgcaacc tgtgaacaga ttgctgactc ccagcatcgg
  481 tctcataggc aaatggtgac aacaaccaat ccactaatca gacatgagaa cagaatggtt
  541 ctagccagca ctacagctaa ggctatggag caaatggctg gatcgagtga gcaagcagca
  601 gaggccatgg atattgctag tcaggccagg caaatggtgc aggcgatgag aaccattggg
  661 actcatccta gctccagtgc tggtctaaaa gatgatcttc ttgaaaattt gcaggcctat
  721 cagaaacgaa tgggggtgca gatgcaacga ttcaagtgat cctctcgtca ttgcagcaaa
  781 tatcattgga atcttgcact tgatattgtg gattcttga
NA of influenza A/WSN/33 (H1N1) virus (SEQ ID NO: 14):
      1 atgaatccaa accagaaaat aataaccatt gggtcaatct gtatggtagt cggaataatt
    61 agcctaatat tgcaaatagg aaatataatc tcaatatgga ttagccattc aattcaaacc
  121 ggaaatcaaa accatactgg aatatgcaac caaggcagca ttacctataa agttgttgct
  181 gggcaggact caacttcagt gatattaacc ggcaattcat ctctttgtcc catccgtggg
  241 tgggctatac acagcaaaga caatggcata agaattggtt ccaaaggaga cgtttttgtc
  301 ataagagagc cttttatttc atgttctcac ttggaatgca ggaccttttt tctgactcaa
  361 ggcgccttac tgaatgacaa gcattcaagg gggaccttta aggacagaag cccttatagg
  421 gccttaatga gctgccctgt cggtgaagct ccgtccccgt acaattcaag gtttgaatcg
  481 gttgcttggt cagcaagtgc atgtcatgat ggaatgggct ggctaacaat cggaatttct
  541 ggtccagatg atggagcagt ggctgtatta aaatacaacg gcataataac tgaaaccata
  601 aaaagttgga ggaagaatat attgagaaca caagagtctg aatgtacctg tgtaaatggt
  661 tcatgtttta ccataatgac cgatggccca agtgatgggc tggcctcgta caaaattttc
  721 aagatcgaga aggggaaggt tactaaatca atagagttga atgcacctaa ttctcactac
  781 gaggaatgtt cctgttaccc tgataccggc aaagtgatgt gtgtgtgcag agacaattgg
  841 cacggttcga accgaccatg ggtgtccttc gaccaaaacc tagattataa aataggatac
  901 atctgcagtg gggttttcgg tgacaacccg cgtcccaaag atggaacagg cagctgtggc
  961 ccagtgtctg ctgatggagc aaacggagta aagggatttt catataagta tggtaatggt
1021 gtttggatag gaaggactaa aagtgacagt tccagacatg ggtttgagat gatttgggat
1081 cctaatggat ggacagagac tgatagtagg ttctctatga gacaagatgt tgtggcaatg
1141 actgatcggt cagggtacag cggaagtttc gttcaacatc ctgagctaac agggctagac
1201 tgtatgaggc cttgcttctg ggttgaatta atcagggggc tacctgagga gaacgcaatc
1261 tggactagtg ggagcatcat ttctttttgt ggtgtgaata gtgatactgt agattggtct
1321 tggccagacg gtgctgagtt gccgttcacc attgacaagt agtttgtt
NP of influenza A/WSN/33 (H1N1) virus (SEQ ID NO: 15):
      1 atggcgacca aaggcaccaa acgatcttac gaacagatgg agactgatgg agaacgccag
    61 aatgccactg aaatcagagc atctgtcgga aaaatgattg atggaattgg acgattctac
  121 atccaaatgt gcaccgaact taaactcagt gattatgagg gacggctgat tcagaacagc
  181 ttaacaatag agagaatggt gctctctgct tttgacgaga ggaggaataa atatctagaa
  241 gaacatccca gtgcggggaa agatcctaag aaaactggag gacctatata caggagagta
  301 gatggaaagt ggaggagaga actcatcctt tatgacaaag aagaaataag acgaatctgg
  361 cgccaagcta ataatggtga cgatgcaacg gctggtctga ctcacatgat gatctggcac
  421 tccaatttga atgatgcaac ttaccagagg acaagagctc ttgttcgcac aggaatggat
  481 cccaggatgt gctcactgat gcagggttca accctcccta ggaggtctgg ggccgcaggt
  541 gctgcagtca aaggagttgg aacaatggtg atggaattga tcagaatgat caaacgtggg
  601 atcaatgatc ggaacttctg gaggggtgag aatggacgga gaacaaggat tgcttatgaa
  661 agaatgtgca acattctcaa agggaaattt caaacagctg cacaaagaac aatggtggat
  721 caagtgagag agagccggaa tccaggaaat gctgagttcg aagatctcat ctttttagca
  781 cggtctgcac tcatattgag agggtcagtt gctcacaagt cctgcctgcc tgcctgtgtg
  841 tatggatctg ccgtagccag tggatacgac tttgaaagag agggatactc tctagtcgga
  901 atagaccctt tcagactgct tcaaaacagc caagtataca gcctaatcag accaaatgag
  961 aatccagcac acaagagtca actggtgtgg atggcatgcc attctgctgc atttgaagat
1021 ctaagagtat caagcttcat cagagggacg aaagtggtcc caagagggaa gctttccact
1081 agaggagttc aaattgcttc caatgaaaac atggagacta tggaatcaag tacccttgaa
1141 ctgagaagca gatactgggc cataaggacc agaagtggag ggaacaccaa tcaacagagg
1201 gcttcctcgg gccaaatcag catacaacct acgttctcag tacagagaaa tctccctttt
1261 gacagaccaa ccattatggc agcattcact gggaatacag aggggagaac atctgacatg
1321 agaaccgaaa tcataaggct gatggaaagt gcaagaccag aagatgtgtc tttccagggg
1381 cggggagtct tcgagctctc ggacgaaaag gcaacgagcc cgatcgtgcc ctcctttgac
1441 atgagtaatg aaggatctta tttcttcgga gacaatgcag aggagtacga caattaaaga
1501 a
NS of influenza A/WSN/33 (H1N1) virus (SEQ ID NO: 16):
      1 atggatccaa acactgtgtc aagctttcag gtagattgct ttctttggca tgtccgcaaa
    61 agagttgcag accaagaact aggtgatgcc ccattccttg atcggcttcg ccgagatcag
  121 aagtccctaa gaggaagagg cagcactctc ggtctggaca tcgaaacagc cacccgtgct
  181 ggaaagcaaa tagtggagcg gattctgaag gaagaatctg atgaggcact caaaatgacc
  241 atggcctctg tacctgcatc gcgctaccta actgacatga ctcttgagga aatgtcaagg
  301 cactggttca tgctcatgcc caagcagaaa gtggcaggcc ctctttgtat cagaatggac
  361 caggcgatca tggataagaa catcatactg aaagcgaact tcagtgtgat ttttgaccgg
  421 ctggagactc taatattact aagggccttc accgaagagg ggacaattgt tggcgaaatt
  481 tcaccactgc cctctcttcc aggacatact gatgaggatg tcaaaaatgc agttggggtc
  541 ctcatcggag gacttgaatg gaataataac acagttcgag tctctgaaac tctacagaga
  601 ttcgcttgga gaagcagtaa tgagaatggg agacctccac tcactccaaa acagaaacgg
  661 aaaatggcgg gaacaattag gtcagaagtt tga
PA of influenza A/WSN/33 (H1N1) virus (SEQ ID NO: 17):
      1 atggaagatt ttgtgcgaca atgcttcaat ccgatgattg tcgagcttgc ggaaaaggca
    61 atgaaagagt atggagagga cctgaaaatc gaaacaaaca aatttgcagc aatatgcact
  121 cacttggaag tgtgcttcat gtattcagat tttcacttca tcgatgagca aggcgagtca
  181 atagtcgtag aacttggcga tccaaatgca cttttgaagc acagatttga aataatcgag
  241 ggaagagatc gcacaatagc ctggacagta ataaacagta tttgcaacac tacaggggct
  301 gagaaaccaa agtttctacc agatttgtat gattacaaga agaatagatt catcgaaatt
  361 ggagtaacaa ggagagaagt tcacatatac tatctggaaa aggccaataa aattaaatct
  421 gagaagacac acatccacat tttctcattc actggggagg aaatggccac aaaggccgac
  481 tacactctcg atgaagaaag cagggctagg atcaaaacca ggctattcac cataagacaa
  541 gaaatggcta gcagaggcct ctgggattcc tttcgtcagt ccgagagagg cgaagagaca
  601 attgaagaaa gatttgaaat cacaggaaca atgcgcaagc ttgccgacca aagtctcccg
  661 ccaaacttct ccagccttga aaattttaga gcctatgtgg atggattcga accgaacggc
  721 tacattgagg gcaagctttc tcaaatgtcc aaagaagtaa atgctagaat tgaacctttt
  781 ttgaaatcaa caccacgacc acttagactt ccggatgggc ctccctgttc tcagcggtcc
  841 aaattcctgc tgatggatgc cttaaaatta agcattgagg acccaagtca tgagggagag
  901 gggataccgc tatatgatgc aatcaaatgc atgagaacat tctttggatg gaaggaaccc
  961 aatgttgtta aaccacacga aaagggaata aatccaaatt atcttctgtc atggaagcaa
1021 gtactggcag aactgcagga cattgagaat gaggagaaaa ttccaaggac taaaaatatg
1081 aagaaaacga gtcagttaaa gtgggcactt ggtgagaaca tggcaccaga aaaggtagac
1141 tttgacgatt gtaaagatgt aggcgatttg aagcaatatg atagtgatga accagaattg
1201 aggtcgcttg caagttggat tcagaatgag ttcaacaagg catgtgaact gaccgattca
1261 agctggatag agctcgatga gattggagaa gatgcggctc caattgaaca cattgcaagc
1321 atgagaagga attatttcac agcagaggtg tctcattgca gagccacaga atacataatg
1381 aagggggtgt acatcaatac tgccttgctt aatgcatcct gtgcagcaat ggatgatttc
1441 caattaattc caatgataag caagtgtaga actaaggagg gaaggcgaaa gaccaatttg
1501 tacggtttca tcataaaagg aagatcccac ttaaggaatg acaccgatgt ggtaaacttt
1561 gtgagcatgg agttttccct cactgaccca agacttgaac cacacaaatg ggagaagtac
1621 tgtgttcttg aggtaggaga tatgcttcta agaagtgcca taggccatgt gtcaaggcct
1681 atgttcttgt atgtgaggac aaatggaacc tcaaaaatta aaatgaaatg ggggatggaa
1741 atgaggcgtt gcctccttca gtcacttcaa caaatcgaga gtatgattga agctgagtcc
1801 tctgtcaagg agaaagacat gaccaaagag ttctttgaaa acaaatcaga aacatggccc
1861 gttggagagt cccccaaagg agtggaggaa ggttccattg ggaaggtctg cagaacttta
1921 ttggcaaagt cggtattcaa cagcttgtat gcatctccac aactggaagg attttcagct
1981 gaatcaagaa aactgcttct tatcgttcag gctcttaggg acaacctgga acctgggacc
2041 tttgatcttg gggggctata tgaagcaatt gaggagtgcc tgattaatga tccctgggtt
2101 ttgcttaatg cttcttggtt caactccttc ctcacacatg cattgagata g
PB1 of influenza A/WSN/33 (H1N1) virus (SEQ ID NO: 18):
      1 atggatgtca atccgacttt acttttctta aaagtgccag cacaaaatgc tataagcaca
    61 actttccctt atactggaga ccctccttac agccatggga caggaacagg atacaccatg
  121 gatactgtca acaggacaca tcagtactca gaaaggggaa gatggacaac aaacaccgaa
  181 actggagcac cgcaactcaa cccgattgat gggccactgc cagaagacaa tgaaccaagt
  241 ggttatgccc aaacagattg tgtattggaa gcaatggcct tccttgagga atcccatcct
  301 ggtatctttg agacctcgtg tcttgaaacg atggaggttg ttcagcaaac acgagtggac
  361 aagctgacac aaggccgaca gacctatgac tggactctaa ataggaacca gcctgctgca
  421 acagcattgg ccaacacaat agaagtgttc agatcaaatg gcctcacggc caatgaatct
  481 ggaaggctca tagacttcct taaggatgta atggagtcaa tgaacaaaga agaaatggag
  541 atcacaactc attttcagag aaagagacga gtgagagaca atatgactaa gaaaatggtg
  601 acacagagaa caataggtaa aaggaagcag agattgaaca aaaggagtta tctaattagg
  661 gcattaaccc tgaacacaat gaccaaagat gctgagagag ggaagctaaa acggagagca
  721 attgcaaccc cagggatgca aataaggggg tttgtatact ttgttgagac actagcaagg
  781 agtatatgtg agaaacttga acaatcagga ttgccagttg gaggcaatga gaagaaagca
  841 aagttggcaa atgttgtaag gaagatgatg accaattctc aggacactga aatttctttc
  901 accatcactg gagataacac caaatggaac gaaaatcaga accctcggat gtttttggcc
  961 atgatcacat atataaccag aaatcagccc gaatggttca gaaatgttct aagtattgct
1021 ccaataatgt tctcaaacaa aatggcgaga ctgggaaagg ggtacatgtt tgagagcaag
1081 agtatgaaac ttagaactca aatacctgca gaaatgctag caagcatcga tttgaaatac
1141 ttcaatgatt caactagaaa gaagattgaa aaaatccggc cgctcttaat agatgggact
1201 gcatcattga gccctggaat gatgatgggc atgttcaata tgttaagtac tgtattaggc
1261 gtctccatcc tgaatcttgg acaaaagaga cacaccaaga ctacttactg gtgggatggt
1321 cttcaatctt ctgatgattt tgctctgatt gtgaatgcac ccaatcatga agggattcaa
1381 gccggagtca acaggtttta tcgaacctgt aagctacttg gaattaatat gagcaagaaa
1441 aagtcttaca taaacagaac aggtacattt gaattcacaa gttttttcta tcgttatggg
1501 tttgttgcca atttcagcat ggagcttccc agctttgggg tgtctgggat caacgagtct
1561 gcggacatga gtattggagt tactgtcatc aaaaacaata tgataaacaa tgatcttggt
1621 ccagcaaccg ctcaaatggc ccttcagctg ttcatcaaag attacaggta cacgtaccgg
1681 tgccatagag gtgacacaca aatacaaacc cgaagatcat ttgaaataaa gaaactgtgg
1741 gagcaaaccc attccaaagc tggactgctg gtctccgacg gaggcccaaa tttatacaac
1801 attagaaatc tccacattcc tgaagtctgc ttgaaatggg aattaatgga tgaggattac
1861 caggggcgtt tatgcaaccc actgaaccca tttgtcaacc ataaagacat tgaatcagtg
1921 aacaatgcag tgataatgcc agcacatggt ccagccaaaa acatggagta tgatgctgtt
1981 gcaacaacac actcctggat ccccaaaaga aatcgatcca tcttgaatac aagccaaaga
2041 ggaatacttg aagatgaaca aatgtaccaa aagtgctgca acttatttga aaaattcttc
2101 cccagcagtt catacagaag accagtcggg atatccagta tggtggaggc tatggtttcc
2161 agagcccgaa ttgatgcacg aattgatttc gaatctggaa ggataaagaa agaggagttc
2221 actgagatca tgaagatctg ttccaccatt gaagagctca gacggcaaaa atag
PB2 of influenza A/WSN/33 (H1N1) virus (SEQ ID NO: 19):
      1 atggaaagaa taaaagaact aaggaatcta atgtcgcagt ctcgcactcg cgagatactc
    61 acaaaaacca ccgtggacca tatggccata atcaagaagt acacatcagg aagacaggag
  121 aagaacccag cacttaggat gaaatggatg atggcaatga aatatccaat tacagcagac
  181 aagaggataa cggaaatgat tcctgagaga aatgagcagg gacaaacttt atggagtaaa
  241 atgaatgacg ccggatcaga ccgagtgatg gtatcacctc tggctgtgac atggtggaat
  301 aggaatggac cagtgacaag tacagttcat tatccaaaaa tctacaaaac ttattttgaa
  361 aaagtcgaaa ggttaaaaca tggaaccttt ggccctgtcc attttagaaa ccaagtcaaa
  421 atacgtcgaa gagttgacat aaatcctggt catgcagatc tcagtgccaa agaggcacag
  481 gatgtaatca tggaagttgt tttccctaac gaagtgggag ccaggatact aacatcggaa
  541 tcgcaactaa cgacaaccaa agagaagaaa gaagaactcc agggttgcaa aatttctcct
  601 ctgatggtgg catacatgtt ggagagagaa ctggtccgca aaacgagatt cctcccagtg
  661 gctggtggaa caagcagtgt gtacattgaa gtgttgcatt tgacccaagg aacatgctgg
  721 gaacagatgt acactccagg aggggaggcg aggaatgatg atgttgatca aagcttaatt
  781 attgctgcta gaaacatagt aagaagagcc acagtatcag cagatccact agcatcttta
  841 ttggagatgt gccacagcac gcagattggt ggagtaagga tggtaaacat ccttaggcag
  901 aacccaacag aagagcaagc cgtggatatt tgcaaggctg caatgggact gagaattagc
  961 tcatccttca gttttggtgg attcacattt aagagaacaa gcggatcatc agtcaagaga
1021 gaggaagagg tgcttacggg caatcttcag acattgaaga taagagtgca tgagggatat
1081 gaagagttca caatggttgg gagaagagca acagctatac tcagaaaagc aaccaggaga
1141 ttgattcagc tgatagtgag tgggagagac gaacagtcga ttgccgaagc aataattgtg
1201 gccatggtat tttcacaaga ggattgtatg ataaaagcag ttagaggtga cctgaatttc
1261 gtcaataggg cgaatcagcg attgaatccc atgcaccaac ttttgagaca ttttcagaag
1321 gatgcaaagg tgctctttca aaattgggga attgaatcca tcgacaatgt gatgggaatg
1381 atcgggatat tgcccgacat gactccaagc accgagatgt caatgagagg agtgagaatc
1441 agcaaaatgg gggtagatga gtattccagc gcggagaaga tagtggtgag cattgaccgt
1501 tttttgagag ttagggacca acgtgggaat gtactactgt ctcccgagga ggtcagtgaa
1561 acacagggaa cagagaaact gacaataact tactcatcgt caatgatgtg ggagattaat
1621 ggtcctgaat cagtgttggt caatacctat cagtggatca tcagaaactg ggaaactgtt
1681 aaaattcagt ggtcccagaa tcctacaatg ctgtacaata aaatggaatt tgagccattt
1741 cagtctttag ttccaaaggc cgttagaggc caatacagtg ggtttgtgag aactctgttc
1801 caacaaatga gggatgtgct tgggacattt gataccgctc agataataaa acttcttccc
1861 ttcgcagccg ctccaccaaa gcaaagtgga atgcagttct cctcattgac tataaatgtg
1921 aggggatcag gaatgagaat acttgtaagg ggcaattctc cagtattcaa ctacaacaag
1981 accactaaaa gactcacagt tctcggaaag gatgctggcc ctttaactga agacccagat
2041 gaaggcacag ctggagttga gtccgcagtt ctgagaggat tcctcattct gggcaaagaa
2101 gacaggagat atggaccagc attaagcata aatgaactga gcaaccttgc gaaaggagag
2161 aaggctaatg tgctaattgg gcaaggagac gtggtgttgg taatgaaacg gaaacggaac
2221 tctagcatac ttactgacag ccagacagcg accaaaagaa ttcggatggc catcaattag
```

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method comprising (a) evolving a parent strain of influenza viral particles in a cell culture comprising human airway cells in the presence of an anti-influenza drug, and (b) isolating drug-resistant progeny influenza viral particles released from the human airway cells.

2. The method of claim 1, wherein step (a) comprises culturing human airway cells comprising the drug-sensitive parent strain of influenza viral particles in a cell culture that comprises an anti-influenza drug for a period of time sufficient to inhibit replication of the influenza viral particles by at least 70%.

3. The method of step 2, wherein the method further comprises culturing human airway cells comprising progeny of the influenza viral particles in a cell culture that comprises the anti-influenza drug.

4. The method of claim 3, wherein the human airway cells comprising progeny of the influenza viral particles are cultured until viral replication increases influenza viral particle number by greater than 50%, relative to baseline.

5. A method comprising (a) culturing human airway cells comprising a drug-sensitive parent strain of influenza viral particles in cell culture that comprises an anti-influenza drug for a period of time sufficient to inhibit replication of a subset of the influenza viral particles, (b) culturing human airway cells comprising progeny of the influenza viral particles in cell culture that comprises the anti-influenza drug, and (c) isolating drug-resistant progeny influenza viral particles released from the human airway cells.

6. The method of any one of claims 2-5, wherein the human airway cells of (a) are cultured in the presence of the anti-influenza drug for a period of time sufficient to inhibit influenza viral particle entry into host cells, their replication in host cells, and/or their release from host cells at least 70%, at least 80%, or at least 90%.

7. The method of any one of claims 1-6 further comprising sequencing viral RNA obtained from the drug-resistant progeny influenza viral particles to identify a drug-resistant strain of influenza virus comprising a mutation in its genome, relative to the parent strain of influenza virus.

8. The method of any one of claims 1-7, wherein step (a) comprises culturing the human airway cells comprising the parent strain of influenza viral particles for a period of time sufficient to enable multiple rounds of viral replication.

9. The method of claim 8, wherein the parent strain of influenza virus is cultured for at least 24 hours, or at least 48 hours.

10. The method of any one of claims 1-9, wherein step (a) comprises passaging the viral particles of the parent strain and/or progeny of the parent strain through successive cultures of the human airway cells, optionally for at least 5, at least 10, at least 15, at least 20, or at least 25 passages, to produce the drug-resistant progeny influenza viral particles.

11. The method of any one of claims 1-10, wherein the drug-resistant progeny influenza viral particles are isolated from drug-resistant virus pools through viral plaque purification.

12. The method of any one of claims 1-11, wherein the anti-influenza virus drug inhibits influenza virus M1, protein, M2 protein, HA protein, or NA protein.

13. The method of any one of claims 1-12, wherein the anti-influenza drug is selected from: oseltamivir (TAMIFLU®), peramivir (RAPIVAB®), zanamivir (RELENZA®), amantadine (SYMMETREL®), rimantadine (FLUMADINE®), and baloxavir marboxil (XOFLUZA®).

14. The method of claim 13, wherein the anti-influenza drug is oseltamivir.

15. The method of claim 14, wherein the anti-influenza drug is amantadine.

16. The method of any one of claims 1-15, wherein the parent influenza virus strain is selected from influenza A/WSN/33 (H1N1), influenza A/Hong Kong/8/68 (H3N2), and influenza A/Avian Influenza (H5N1).

17. The method of any one of claims 1-16, wherein the anti-influenza drug is present in the cell culture at a concentration of 0.1 μM to 10 μM, or 0.5 μM to 2 μM.

18. The method of any one of claims 1-17, wherein step (a) comprises evolving two or more parent strains of influenza virus in the same cell culture comprising human airway cells in the presence of the anti-influenza drug.

19. The method of any one of claims 1-18 further comprising developing a vaccine or other immunogenic composition against the drug-resistant strain of influenza virus.

20. The method of any one of claims 1-19, wherein the vaccine is selected from live-attenuated virus vaccines, inactivated viral vaccines, recombinant viral vaccines, polypeptide vaccines, DNA vaccines, RNA vaccines, and virus-like particles.

21. The method of any one of claims 1-20, wherein the human airway cells are human lung cells, optionally human lung epithelial cells.

22. The method of any one of claims 1-21, wherein human airway cells are component of a microfluidic device.

23. An immunogenic composition comprising an influenza virus matrix 2 (M2) antigen variant that comprises an amino acid substitution at position 31 and an amino acid substitution at position 34, relative to a H1N1 influenza virus M2 antigen, wherein the H1N1 influenza virus M2 antigen comprises the amino acid sequence of SEQ ID NO: 3.

24. An immunogenic composition comprising an influenza virus matrix 2 (M2) antigen variant that comprises an amino acid substitution at position 31 and an amino acid substitution at position 46, relative to a H1N1 influenza virus M2 antigen, wherein the H1N1 influenza virus M2 antigen comprises the amino acid sequence of SEQ ID NO: 3.

25. The immunogenic composition of claim 23 or 24, wherein the amino acid substitution at position 31 is S31N.

26. The immunogenic composition of claim 23 or 25, wherein the amino acid substitution at position 34 is G34E.

27. The immunogenic composition of claim 24 or 25, wherein the amino acid substitution at position 46 is L46P.

28. The immunogenic composition of claim 26, wherein the influenza virus M2 antigen variant comprises an amino acid sequence that is at least 95% identical to the amino acid sequence of SEQ ID NO: 1.

29. The immunogenic composition of claim 28, wherein the influenza virus M2 antigen variant comprises the amino acid sequence of SEQ ID NO: 1.

30. The immunogenic composition of claim 27, wherein the influenza virus M2 antigen variant comprises an amino acid sequence that is at least 95% identical to the amino acid sequence of SEQ ID NO: 1.

31. The immunogenic composition of claim 30, wherein the influenza virus M2 antigen variant comprises the amino acid sequence of SEQ ID NO: 2.

32. A method comprising administering to a subject the immunogenic composition of any one of claims 23-31 in an effective amount to induce in the subject an antigen-specific immune response.
